(19) United States
(12) Patent Application Publication
(10) Pub. No.: US 2005/0180959 A1
(43) Pub. Date: Aug. 18, 2005

(54) Kinases involved in the regulation of energy homeostasis

(76) Inventors: Karsten Eulenberg, Gottingen (DE); Gunter Bronner, Gottingen (DE)

Correspondence Address: Christopher B Linder;Thomas Kayden Horstemeyer & Risley, Suite 1750, 100 Galleria Parkway NW, Atlanta, GA, 30339-5948, US

(21) Appl. No.: 10/503,870

(22) PCT Filed: Feb. 6, 2003
(86) PCT No.: PCT/EP03/01189

(51) Int. Cl. A61K 48/00; C12Q 1/68

(52) U.S. Cl. 424/93.21; 514/44; 435/6

(57) ABSTRACT

The present invention discloses Inositol hexakisphosphate kinase or RYK kinase homologous proteins regulating the energy homeostasis and the metabolism of energy storage metabolites, and polynucleotides, which identify and encode the proteins disclosed in this invention. The invention also relates to the use of these sequences in the diagnosis, study, prevention, and treatment of metabolic diseases and disorders.

This invention relates to the use of nucleic acid sequences encoding Inositol hexakisphosphate kinase or RYK kinase or homologous proteins, and the polypeptides encoded thereby and to the use of these sequences or effectors of Inositol hexakisphosphate kinase or RYK kinase nucleic acids or polypeptides, particularly inhibitors or activators, in the diagnosis, study, prevention, and treatment of diseases and disorders related to body-weight regulation, for example, but not limited to, metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly understood human disease that becomes more and more relevant for western society. Obesity is defined as an excess of body fat, frequently resulting in a significant impairment of health. Cardiovascular risk factors like hypertension, high blood levels of triglycerides and fasting glucose as well as low blood levels of HDL cholesterol are often linked to obesity. This typical cluster of symptoms is commonly defined as “metabolic syndrome” (Reaven, 2002, Circulation 106(3): 286-8 reviewed). The metabolic syndrome often precedes the development of type II diabetes and cardiovascular disease (McCook, 2002, JAMA 288:2709-2716). Human obesity is strongly influenced by environmental and genetic factors, whereby the environmental influence is often a hurdle for the identification of (human) obesity genes. Obesity is influenced by genetic, metabolic, biochemical, psychological, and behavioral factors. As such, it is a complex disorder that must be addressed on several fronts to achieve lasting positive clinical outcome.

The molecular factors regulating food intake and body weight balance are incompletely understood. Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity (Friedman J. M. and Leibel R. L., (1992) Cell 69(2): 217-220). In the ob mouse a single gene mutation (obese) results in profound obesity, which is accompanied by diabetes (Friedman J. M. et al., (1991) Genomics 11: 1054-1062).

Insulin resistance greatly increases the risk of developing the metabolic syndrome (Reaven, 2002, Circulation 106(3): 286-8 reviewed). The metabolic syndrome often precedes the development of type II diabetes and cardiovascular disease (McCook, 2002, JAMA 288: 2709-2716). The control of blood lipid levels and blood glucose levels is the essential for the treatment of the Metabolic Syndrome (see, for example, Santomauro A. T. et al., (1999) Diabetes, 48(9): 1836-1841). Insulin amongst other hormones plays a key role in the regulation of the fuel metabolism. High blood glucose levels stimulate the secretion of insulin by pancreatic beta-cells. Insulin leads to the storage of glycogen and triglycerides and to the synthesis of proteins. The entry of glucose into muscles and adipose cells is stimulated by insulin. In patients who suffer from diabetes mellitus either the amount of insulin produced by the pancreatic islet cells is to low (Diabetes Type 1 or insulin dependent diabetes mellitus IDDM) or liver and muscle cells loose their ability to respond to normal blood insulin levels (insulin resistance). In the next stage pancreatic cells become unable to produce sufficient amounts of insulin (Diabetes Type II or non insulin dependent diabetes mellitus NIDDM).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses a specific gene involved in the regulation of body-weight, energy homeostasis, metabolism, and obesity, and thus in disorders related thereto such as metabolic syndrome, eating disorder, cachexia, diabetes mellitus, hypertension, coronary heart disease, hypercholesterolemia, dyslipidemia, osteoarthritis, and gallstones. The present invention describes the human Inositol hexakisphosphate kinase (3, 1, or 2) or RYK kinase gene as being involved in those conditions mentioned above.

Reversible transfer of phosphate groups to substrates as proteins, lipids or other organic compounds is the main strategy for controlling activities of eukaryotic cells. Many known signal transduction pathways are cascades of phosphate donor and acceptor molecules that are modulated in function by the state of phosphorylation. Kinases are enzymes that transfer phosphate groups to specific substrates.

Inositol (1,4,5) trisphosphate is a messenger molecule that releases calcium from intracellular stores. Polyphosphate homologues with up to eight phosphate groups have been identified, including pyrophosphates. Inositol pyrophosphates are formed by several enzymes including Inositol hexakisphosphate kinases. Members of the inositol hexakisphosphate kinase family phosphorylate inositol hexakisphosphate to diphosphoinositol pentakisphosphate, which is a ‘high energy’ candidate of cell trafficking. ‘High-energy’ pyrophosphates may play important roles in protein phosphorylation. In mammalians (human and mouse), three inositol hexakisphosphate kinases are known. Inositol hexakisphosphate kinase 1 (IHKPK1) and inositol hexakisphosphate kinase 2 (IHKPK2) phosphorylate inositol hexakisphosphate (IHKP) to diphosphoinositol pentakisphosphate, a candidate regulator of cellular trafficking (Saiardi A. et al., (2000) J Biol Chem 275(32):24686-24692). IHKPK2 has been described as positive regulator of apoptosis and mediates growth suppressive and apoptotic effects of interferon-beta in ovarian carcinoma cells (Morrison B. H. et al., (2001) J Biol Chem 276(27):24965-14970). IHKPK3 displays a more basic character than the other two enzymes IHKPK1 and IHKPK2. IHKPK3 is most enriched in the brain where its localization resembles IHKPK1 and IHKPK2. Intracellular disposition discriminates the three enzymes with IHKPK2 being exclusively nuclear, IHKPK3 predominating in the cytoplasm, and IHKPK1 displaying comparable nuclear and cytosolic densities (Saiardi A. et al., (2001) J Biol Chem 276(42):39179-39185).

The Drosophila gene doughnut on 2 (dnt) encodes for a receptor tyrosine kinase-like protein that is 70% identical to the Drosophila Derailed (RYK) protein. Dnt is expressed in invaginating cells during embryogenesis in Drosophila (Savant-Bhonsale, S. et al., 1999, Gene 231(1-2): 155-61). As shown in this invention, the Drosophila dnt kinase is most homologous to Drosophila drl and human tyrosine-protein kinase RYK precursor, herein referred to as RYK kinase.

RYK kinase is an atypical member of the family of growth factor receptor protein tyrosine kinases, having different activation and nucleotide binding domains. This kinase belongs to a subfamily whose members do not appear to be regulated by phosphorylation in the activation segment. A RYK kinase ligand has not been identified so far. The protein does not show detectable autophosphorylation activity in vitro but is capable to activate the MAPK pathway (Katso, R. M., 1999, MCB19 (9): 6427-40).

So far, it has not been described that Inositol hexakisphosphate kinase (3, 1, or 2) or RYK kinase protein is involved in the regulation of energy homeostasis and body-weight regulation and related disorders, and thus, no functions in metabolic diseases and other diseases as listed above have been discussed. In this invention we demonstrate that the correct gene dose of Inositol hexakisphosphate kinase or RYK kinase is essential for maintenance of energy homeostasis. A genetic screen was used to identify that mutation of a Inositol hexakisphosphate kinase or RYK kinase homologous gene causes obesity, reflected by a significant increase of triglyceride content, the major energy storage substance.

Polynucleotides encoding a protein with homologies to Inositol hexakisphosphate kinase or RYK kinase are suitable to investigate diseases and disorders as described above. Further new compositions useful in diagnosis, treatment, and prognosis of diseases and disorders as described above are provided.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies, which are reported in the publications which might be used in connection with the invention. Nothing herein is to be construed as an admission that the invention is not entitled to antedate such disclosure.

The present invention discloses that Inositol hexakisphosphate kinase or RYK kinase homologous proteins are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences in the diagnosis, study, prevention, and treatment of diseases and disorders, for example, but not limited to, metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones.

The term “polynucleotide comprising the nucleotide sequence as shown in GenBank Accession number” relates to the expressible gene of the nucleotide sequences deposited under the corresponding GenBank Accession number. The term “GenBank Accession Number” relates to NCBI GenBank database entries (Ref.: Benson et al., (2000) Nucleic Acids Res. 28: 15-18).

Inositol hexakisphosphate kinase or RYK kinase homologous proteins and nucleic acid molecules coding therefore are obtainable from insect or vertebrate species, e.g. mammals or birds. Particularly preferred are homologous nucleic acids, particularly nucleic acids encoding human inositol hexakisphosphate kinase 3, human inositol hexakisphosphate kinase 1, human inositol hexakisphosphate kinase 2, or a human Drl kinase.

The invention particularly relates to a nucleic acid molecule encoding a polypeptide contributing to regulating the energy homeostasis and the metabolism of triglycerides, wherein said nucleic acid molecule comprises
(a) the nucleotide sequence of (i) GadFly Accession Number CG10082, or inositol hexakisphosphate kinase 3 (SEQ ID NO: 1), inositol hexakisphosphate kinase 1 (SEQ ID NO: 3), or inositol hexakisphosphate kinase 2 (SEQ ID NO: 5), or (ii) GadFly Accession Number CG17559, or human Drl kinase (SEQ ID NO: 9), and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of Inositol hexakisphosphate kinase or RYK kinase protein, preferably inositol hexakisphosphate kinase 3 (SEQ ID NO:2), inositol hexakisphosphate kinase 1 (SEQ ID NO: 4), inositol hexakisphosphate kinase 2 (SEQ ID NO: 6), and/or of human RYK kinase (SEQ ID NO:10),
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases.

The invention is based on the finding that Inositol hexakisphosphate kinase or RYK kinase homologous proteins and the polynucleotides encoding these, are involved in the regulation of triglyceride storage and therefore energy homeostasis. The invention describes the use of these polypeptides or fragments thereof, polynucleotides or fragments thereof and effectors (receptors) of these molecules, e.g. antibodies, biologically active nucleic acids, such as antisense molecules, RNAi molecules or ribozymes, aptamers, peptides or low-molecular weight organic compounds recognizing said polynucleotides or polypeptides for the diagnosis, study, prevention, or treatment of diseases and disorders related thereto, including metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones.

Accordingly, the present invention relates to genes with novel functions in body-weight regulation, energy homeostasis, metabolism, and obesity. To find genes with novel functions in energy homeostasis, metabolism, and obesity, a functional genetic screen was performed with the model organism Drosophila melanogaster (Meigen). The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syncrome including obesity) and the modified expression of a fly gene can identify the association of the human ortholog with the particular human disease.

In one embodiment, a forward genetic screen is performed in fly displaying a mutant phenotype due to misexpression of a known gene (see, Johnston Nat Rev Genet 3: 176-188 (2002); Rorth P., (1996) Proc Natl Acad Sci USA 93: 12418-12422). In this invention, we have used a genetic screen to identify gene mutations that cause canges in the body weight which is reflected by a significant change of triglyceride levels. Resources for screening were a Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells, and obese people mainly show a significant increase in the content of triglycerides. In order to isolate genes with a function in energy homeostasis, several thousand EP-lines were tested for their triglyceride content after a prolonged feeding period (see Examples for more detail). Lines with significantly changed triglyceride content were selected as positive candidates for further analysis. Additionally, glycogen levels were analysed.

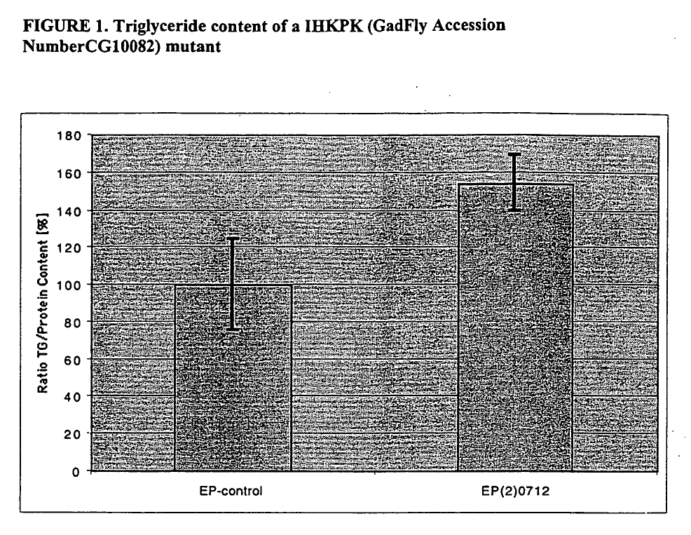

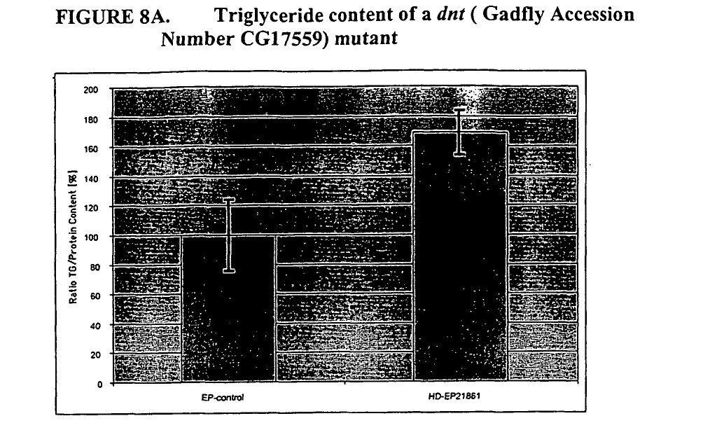

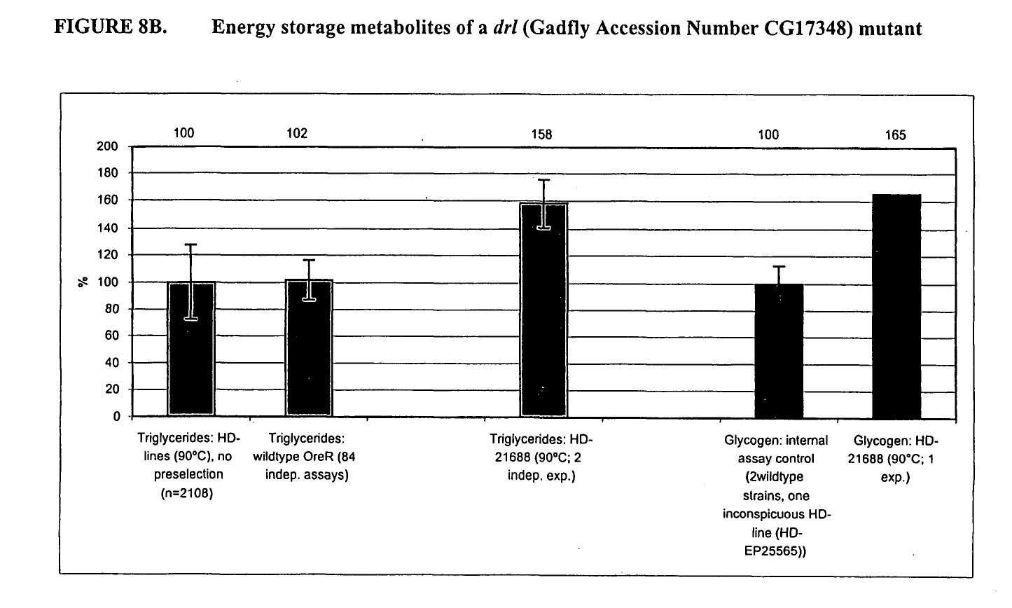

In one embodiment, male flies homozygous for the integration of vectors for Drosophila lines EP(2)0712 and HD-EP(2)21861, respectively, were analyzed in an assay measuring the triglyceride contents of these flies, illustrated in more detail in the EXAMPLES section of this invention. The results of the triglyceride content analysis are shown in FIG. 1 and FIG. 8A, respectively. The average triglyceride level of the fly collection in which the fly line was found is shown as 100% in FIG. 1 and FIG. 8A (first column, EP-control). The average increase of triglyceride content of the homozygous viable Drosophila line EP(2)0712 (referred to as ‘EP(2)0712’ in this invention) is 50% (see FIG. 1, second column, ‘EP(2)0712’). The average increase of triglyceride content of the homozygous viable Drosophila line HD-EP(2)21861 (referred to as ‘HD-EP(2)21861’ in this invention) is 70% (see FIG. 8A, second column, ‘HD-EP(2)21861’). It was found in this invention that homozygous EP(2)0712 flies and HD-EP(2)21861 flies have a significant higher triglyceride content than the control flies tested. The increase of triglyceride content due to the loss of a gene function suggests gene activities in energy homeostasis in a dose dependent manner that controls the amount of energy stored as triglycerides.

These results were confirmed by further findings obtained by studying a Drosophila Gadfly accession number CG17348 derailed (drl) mutant. drl is the second RYK-ortholog in Drosophila and we found, that this mutant displays the same metabolic phenotype as the dnt mutant, confirming a conserved metabolic function of ryk-homolog proteins in Drosophila. In addition to an increased triglyceride content, the glycogen content is elevated due to the loss of a gene function (see FIG. 8B). Glycogen is a large branched polymer of glucose residues that is mainly stored in liver and muscle cells. Glycogen synthesis and degradation is central to the control of the blood glucose level. The results are clearly indicating a role of drl in energy homeostasis and glucose regulation.

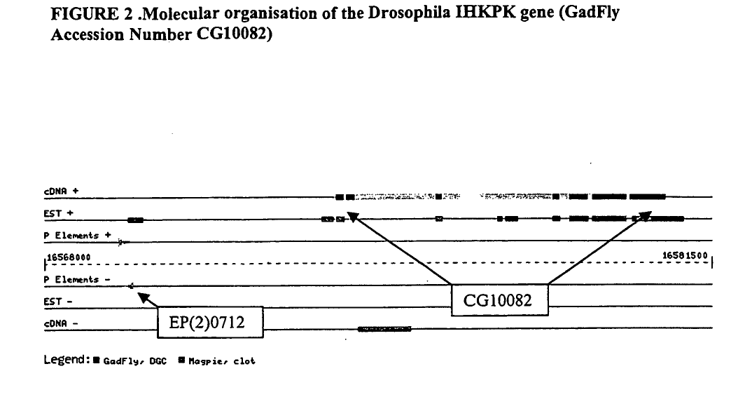

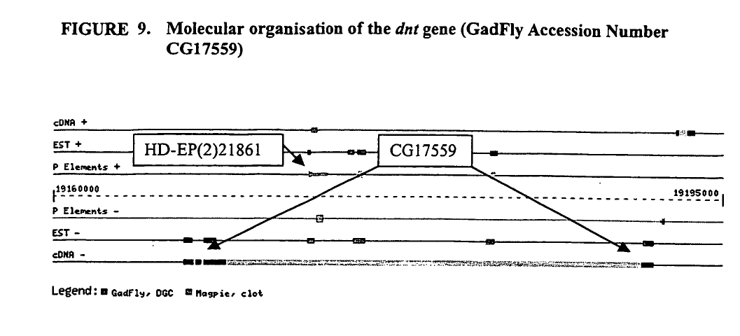

Nucleic acids encoding the Inositol hexakisphosphate kinase or RYK kinase proteins of the present invention were identified using a plasmid-rescue or iPCR technique. Genomic DNA sequences for the inositol hexakisphosphate kinase homologous gene were isolated that are localised adjacent to the EP vector. Using those isolated genomic sequences public databases like Berkeley Drosophila Genome Project (GadFly) were screened thereby confirming the homozygous viable integration site of the vectors. For example, EP(2)0712 vector is integrated 5′ into the cDNA of the Drosophila gene in antisense orientation, identified as Berkeley Drosophila Genome Project Accession No. CG10082 (FIG. 2). In another example, HD-EP(2)21861 vector is integrated into the first large intron of doughnut on 2 (dnt) gene in antisense orientation, identified as Berkeley Drosophila Genome Project Accession No. CG17559 (FIG. 9). FIG. 2 and FIG. 9 show the molecular organisation of these gene loci. The chromosomal localization site of the integration of the vector of EP(2)0712 is at gene locus 2R, 57F6 and of the vector of HD-EP(2)21861 is at gene locus 2L, 37D4-37D6. In FIG. 2 and FIG. 9, genomic DNA sequence is represented as a black dotted line in the middle that includes the integration site of the vectors. Numbers represent the coordinates of the genomic DNA. Grey bars on the two “cDNA”-lines represent the predicted genes (as predicted by the Berkeley Drosophila Genome Project, GadFly and by Magpie). Predicted exons of the Drosophila cDNA are shown as dark grey bars and predicted introns as light grey bars.

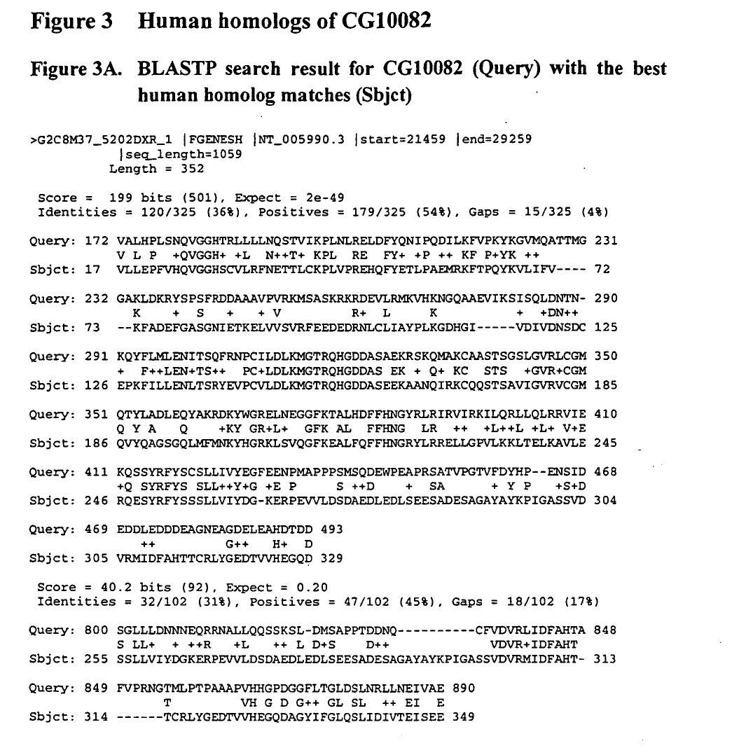

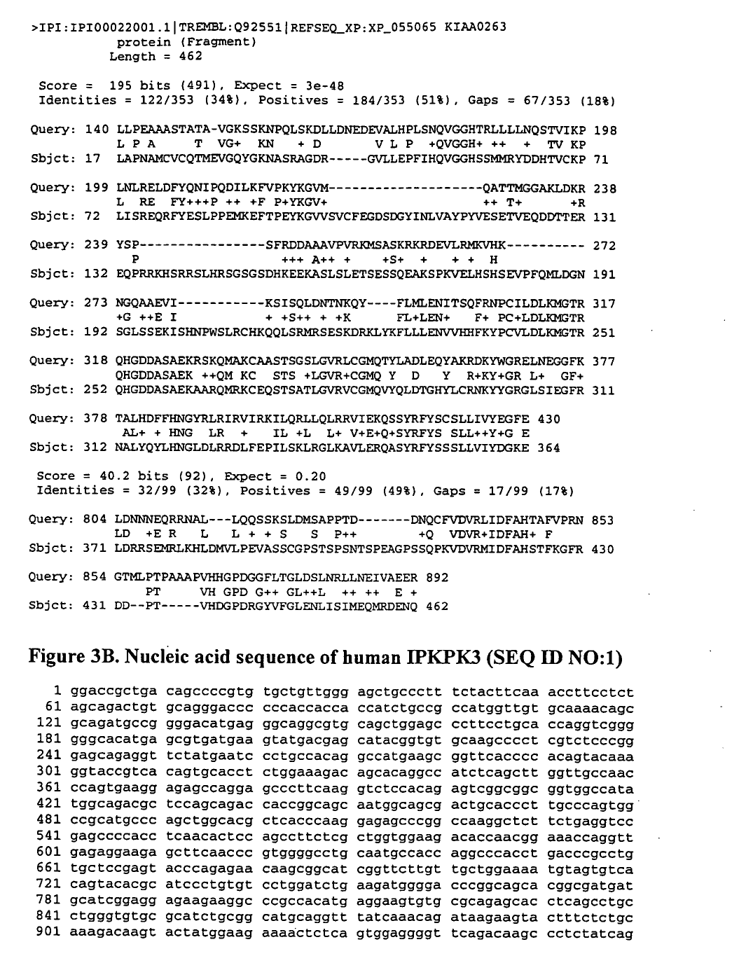

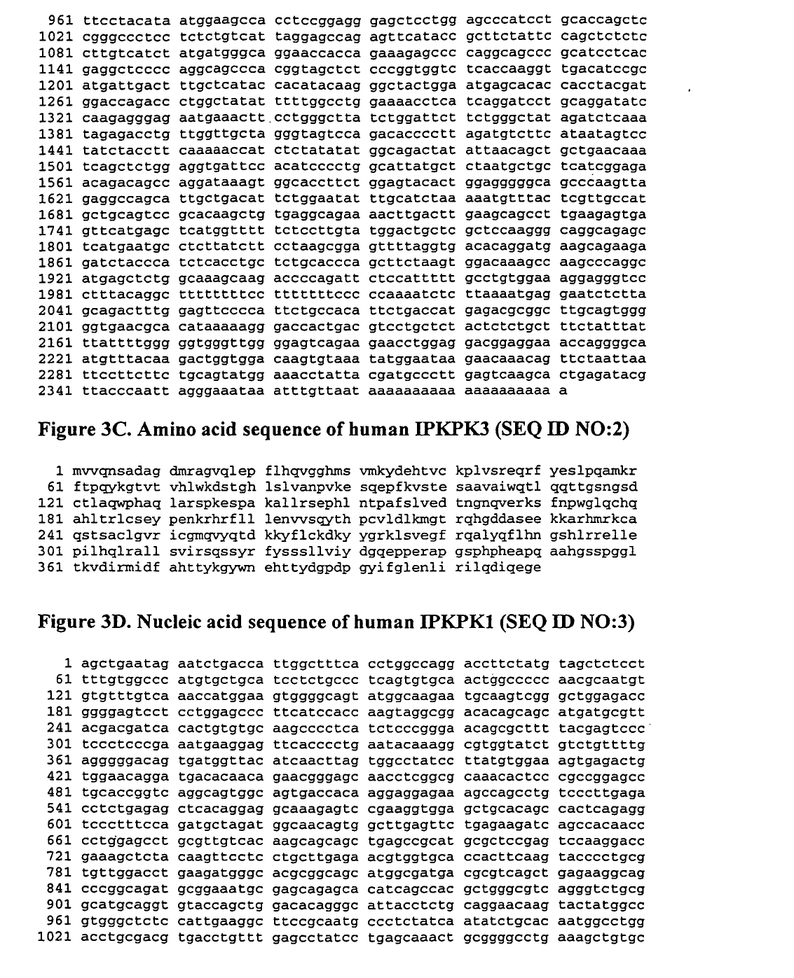

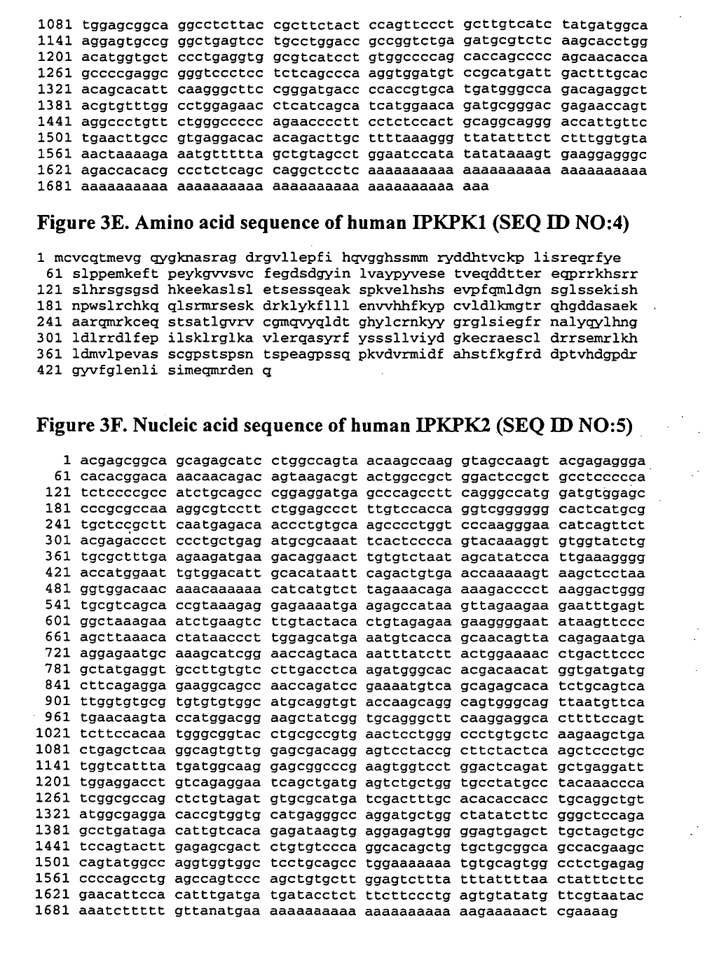

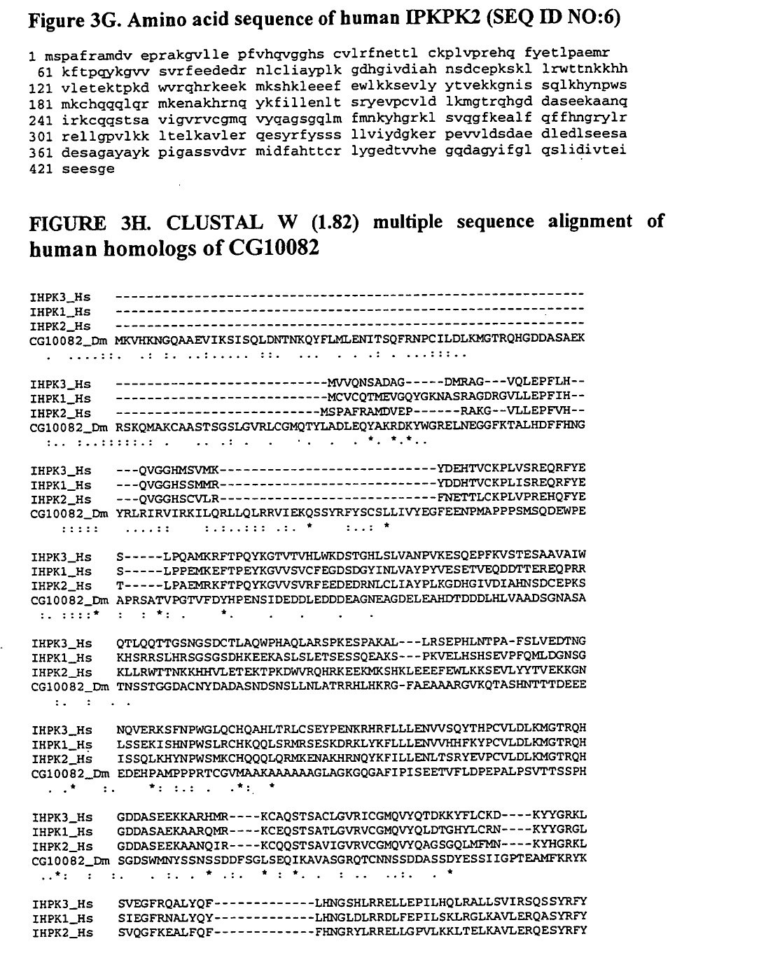

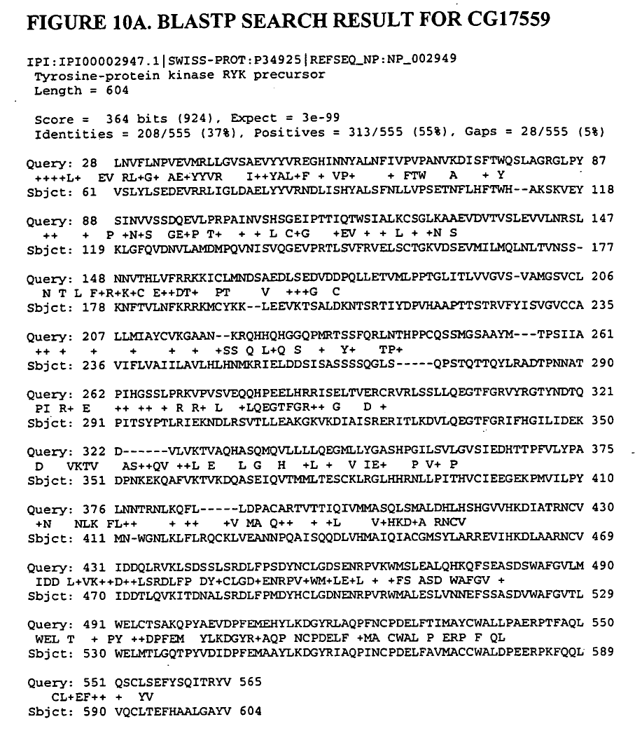

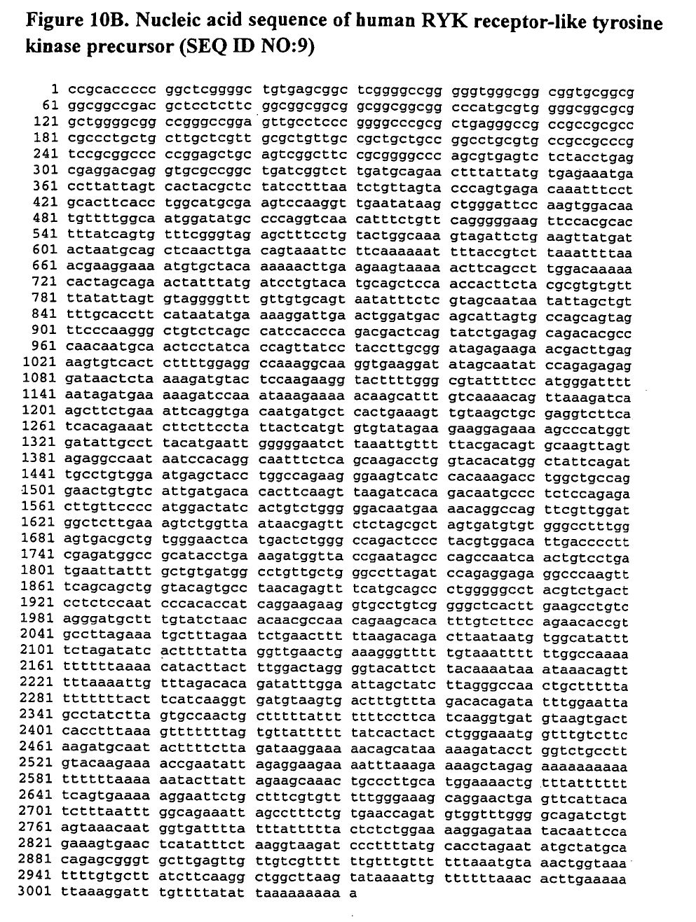

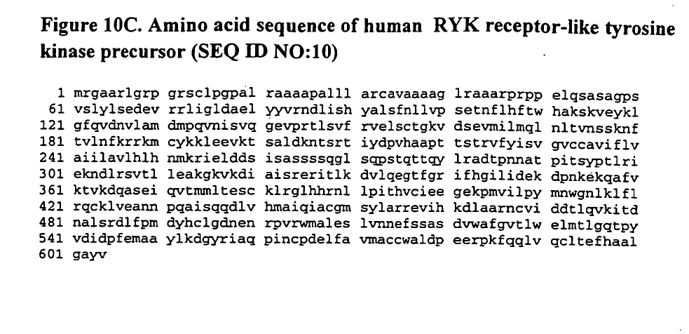

The Drosophila genes and proteins encoded thereby with functions in the regulation of triglyceride metabolism were further analysed in publicly available sequence databases (see EXAMPLES for more detail) and mammalian homologs were identified (see FIGS. 3A and 3H and FIG. 10A).

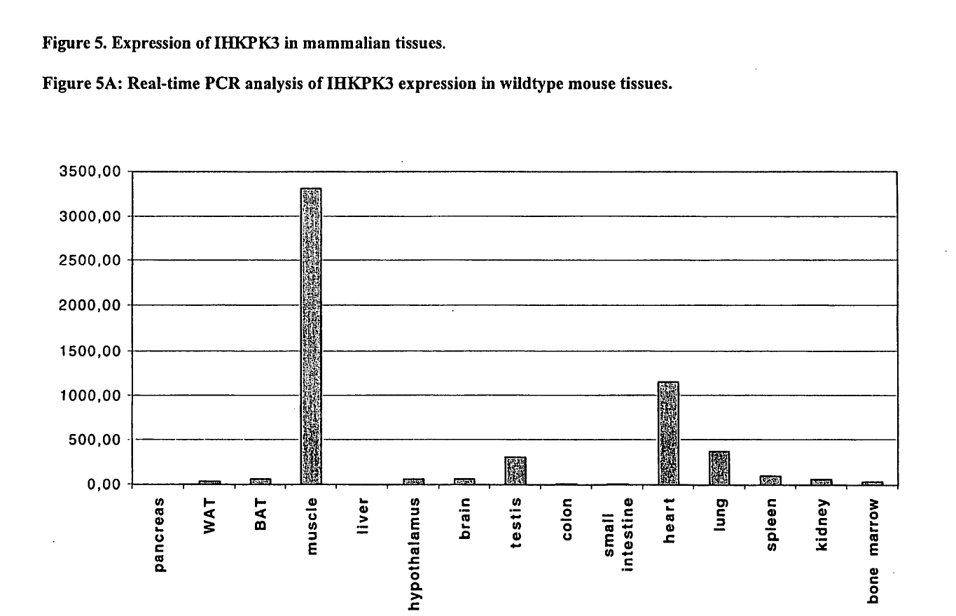

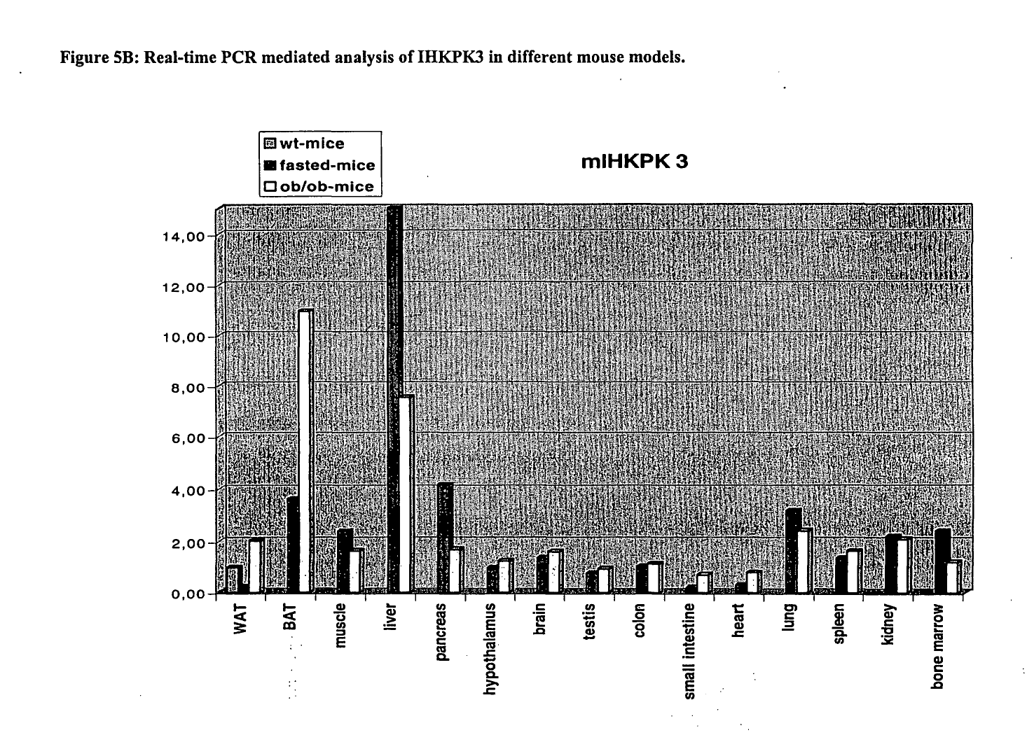

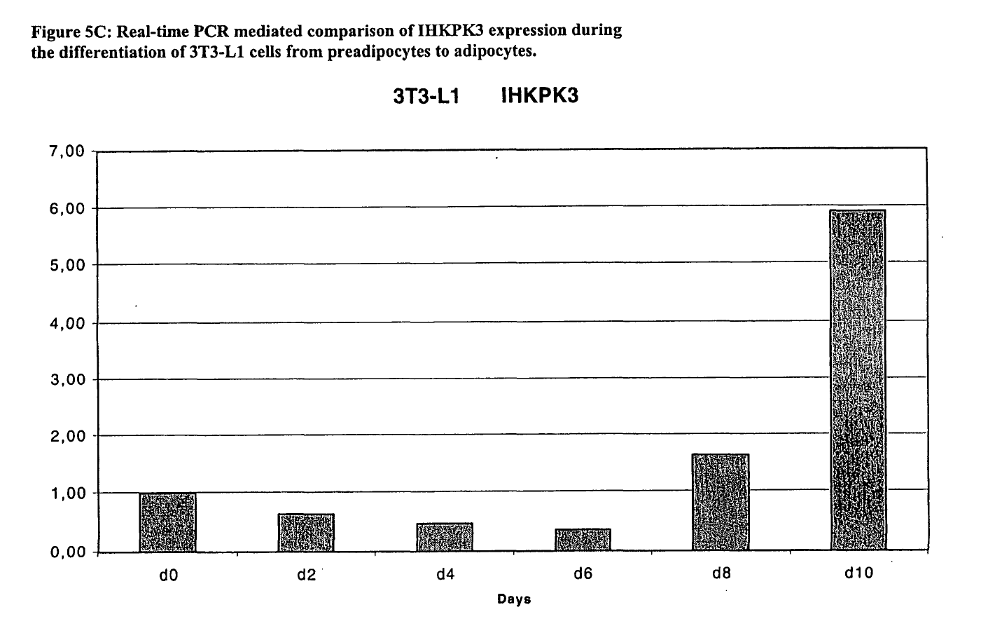

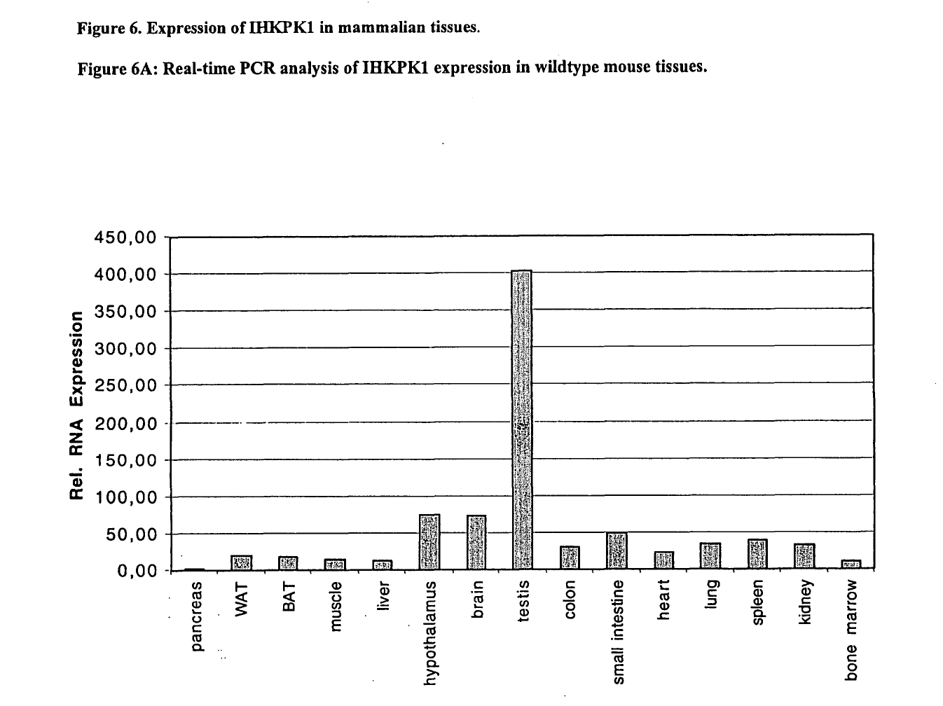

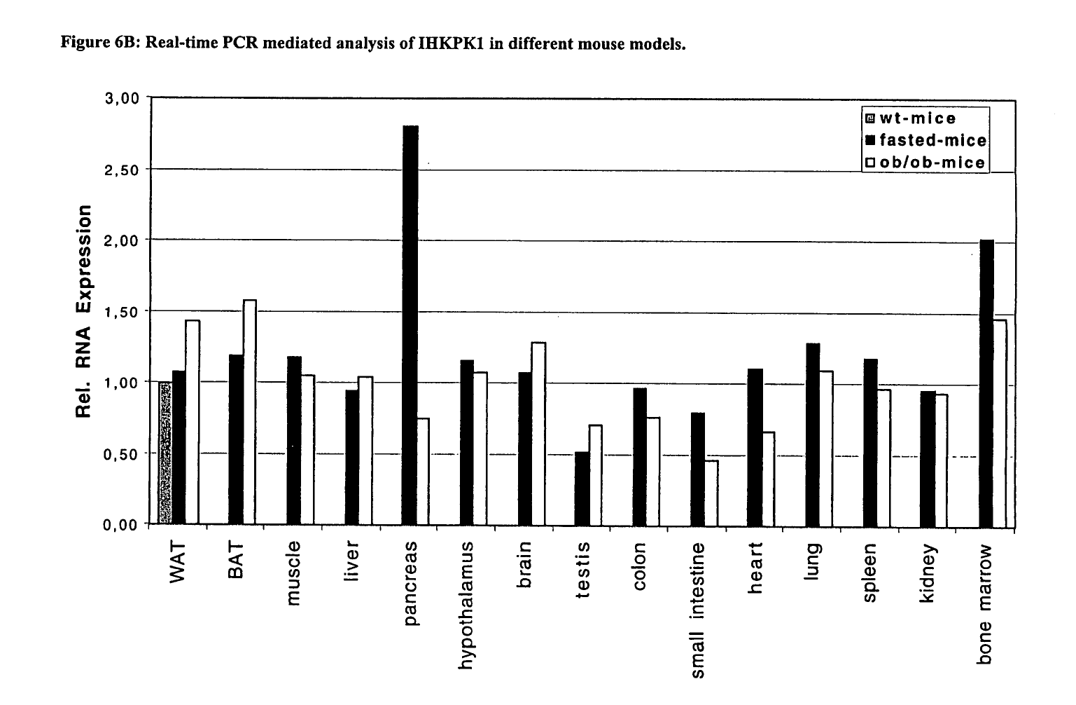

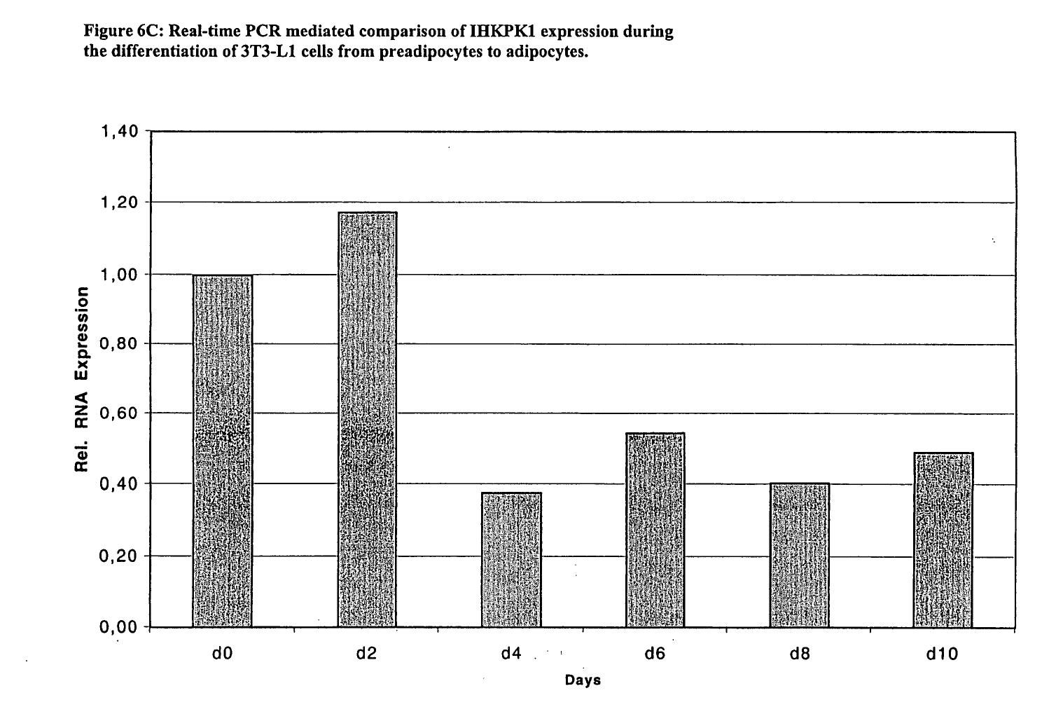

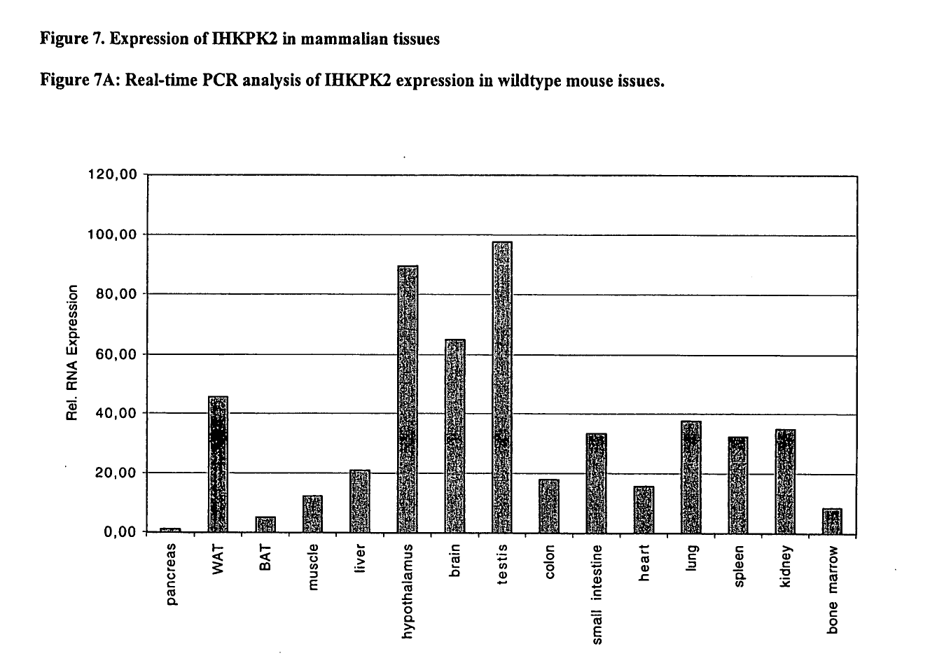

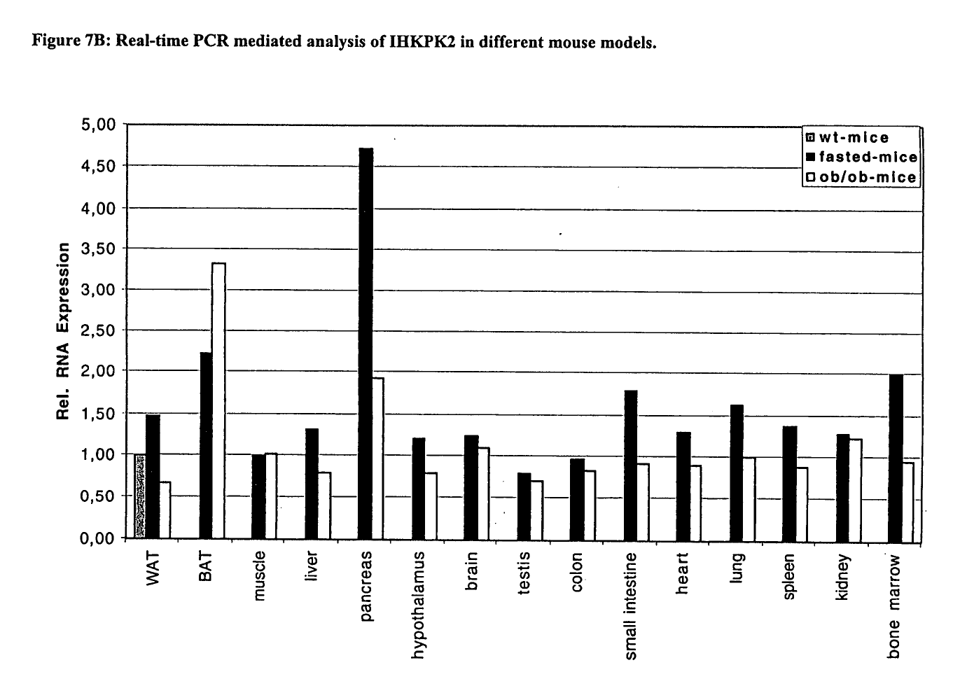

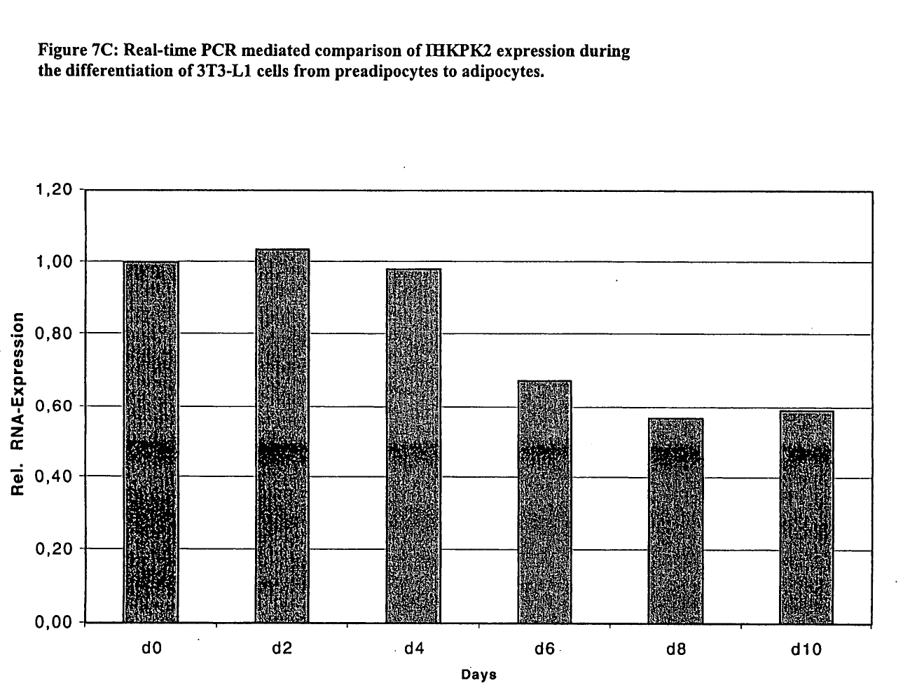

The function of the mammalian homologs in energy homeostasis was further validated in this invention by analyzing the expression of the transcripts in different tissues and by analyzing the role in adipocyte differentiation. Expression profiling studies (see Examples for more detail) confirm the particular relevance of the protein(s) of the invention as regulators of energy metabolism in mammals. For example, transcripts of IHKPK2 and IHKPK1 are more restricted in neuronal tissues and testis of mammals (FIGS. 6A and 7A, respectively). IHKPK3 transcripts show high expression in muscle and heart tissues (FIG. 5A). In addition, IHKPK1 and IHKPK3 are also clearly expressed in white adipose tissue (WAT) and brown adipose tissue (BAT); IHKPK2 shows high levels of expression especially in WAT (see FIG. 5A, FIG. 6A, FIG. 7A, respectively). Brown adipose tissue is a well characterized tissue which is well developed in newborn mammals, including humans. One important task of BAT is to generate heat and maintain body temperature homeostasis in newborn. Thus an expression of the protein of the invention in adipose tissues is confirming a role in the regulation of energy homeostasis and thermogenesis.

Further, we show that the proteins of the invention are regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569). We found, for example, that the expression of IHKPK1 and IHKPK2 is strongly upregulated in the pancreas of fasted mice (see FIG. 6B and FIG. 7B, respectively). The expression of IHKPK3 is strongly upregulated in liver of fasted mice (FIG. 5B). In addition, a marked upregulation of IHKPK3, IHKPK1, and IHKPK2 can be observed in the metabolically active tissue (for example, brown adipose tissue (BAT)) of genetically obese (ob/ob) as well as of fasted mice (see FIG. 5B, FIG. 6B, and FIG. 7B, respectively). IHKPK3 is downregulated in white adipose tissue (WAT) of fasted mice (see FIG. 5B), supporting a hypothesis that the protein of the invention is a modulator of adipogenesis.

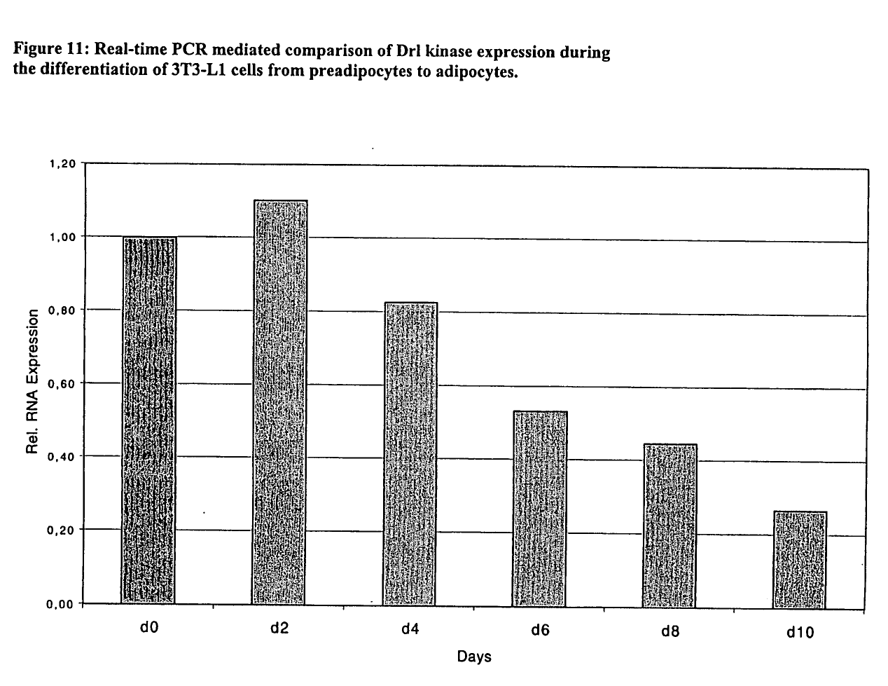

In addition, we show in this invention that the mRNA of IHKPK1 and IHKPK2 and also RYK kinase is significantly down-regulated during adipocyte differentiation in vitro (see EXAMPLES for more detail, see FIG. 6C and FIG. 6C and FIG. 11), suggesting a role as modulator of adipocyte lipid accumulation. With regard to changes in expression intensity during the differentiation of preadipocytes to adipocytes, a strong increase in relative signal intensity can be observed for IHKPK3 expression during the in vitro differentiation program of 3T3-L1 cells (see FIG. 5C). Thus, we conclude that the protein of the invention (or variants thereof) have a function in the metabolism of mature mammalian adipocytes.

The invention also encompasses polynucleotides that encode Inositol hexakisphosphate kinase or RYK kinase or homologous proteins. Accordingly, any nucleic acid sequence, which encodes the amino acid sequences of Inositol hexakisphosphate kinase or RYK kinase can be used to generate recombinant molecules that express Inositol hexakisphosphate kinase or RYK kinase. In a particular embodiment, the invention encompasses the polynucleotide of (i) Drosophila CG10082, or human IHKPK3, human IHKPK1, or human IHKPK2, or (ii) Drosophila dnt CG17559, or human RYK kinase. It will be appreciated by those skilled in the art that as a result of the degeneracy of the genetic code, a multitude of nucleotide sequences encoding Inositol hexakisphosphate kinase or RYK kinase, some bearing minimal homology to the nucleotide sequences of any known and naturally occurring gene, may be produced. Thus, the invention contemplates each and every possible variation of nucleotide sequence that could be made by selecting combinations based on possible codon choices.

Also encompassed by the invention are polynucleotide sequences that are capable of hybridising to the claimed nucleotide sequences, and in particular, those of the polynucleotide encoding (i) Drosophila CG10082, and/or human IHKPK3, IHKPK1, or IHKPK2, or (ii) Drosophila dnt CG17559, and/or human RYK kinase, under various conditions of stringency. Hybridisation conditions are based on the melting temperature (Tm) of the nucleic acid-binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding Inositol hexakisphosphate kinase or RYK kinase which are encompassed by the invention include deletions, insertions, or substitutions of different nucleotides resulting in a polynucleotide that encodes the same or a functionally equivalent Inositol hexakisphosphate kinase or RYK kinase.

The encoded proteins may also contain deletions, insertions, or substitutions of amino acid residues, which produce a silent change and result in a functionally equivalent Inositol hexakisphosphate kinase or RYK kinase. Deliberate amino acid substitutions may be made on the basis of similarity in polarity, charge, solubility, hydrophobicity, hydrophilicity, and/or the amphipathic nature of the residues as long as the biological activity of Inositol hexakisphosphate kinase or RYK kinase is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding Inositol hexakisphosphate kinase or RYK kinase. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

The nucleic acid sequences encoding Inositol hexakisphosphate kinase or RYK kinase may be extended utilising a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and libraries to walk in genomic DNA (Clontech, Palo Alto, Calif.). This process avoids the need to screen libraries and is useful in finding intron/exon junctions.

In order to express a biologically active Inositol hexakisphosphate kinase or RYK kinase, the nucleotide sequences encoding Inositol hexakisphosphate kinase or RYK kinase or functional equivalents, may be inserted into appropriate expression vectors, i.e. a vector, which contains the necessary elements for the transcription and translation of the inserted coding sequence. Methods, which are well known to those skilled in the art, may be used to construct expression vectors containing sequences encoding Inositol hexakisphosphate kinase or RYK kinase and appropriate transcriptional and translational control elements. These methods include in vitro recombinant DNA techniques. synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilised to contain and express sequences encoding Inositol hexakisphosphate kinase or RYK kinase. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g. baculovirus); plant cell systems transformed with virus expression vectors (e.g. cauliflower mosaic virus, CaMV; tobacco mosaic virus, TMV) or with bacterial expression vectors (e.g. Ti or PBR322 plasmids); or animal cell systems. The “control elements” or “regulatory sequences” are those non-translated regions of the vector-enhancers, promoters, 5′ and 3′ untranslated regions which interact with host cellular proteins to carry out transcription and translation. Such elements may vary in their strength and specificity. Depending on the vector system and host utilised, any number of suitable transcription and translation elements, including constitutive and inducible promoters, may be used.

The presence of polynucleotide sequences encoding Inositol hexakisphosphate kinase and/or RYK kinase can be detected by DNA-DNA or DNA-RNA hybridisation or amplification using probes or portions or fragments of polynucleotides encoding Inositol hexakisphosphate kinase or RYK kinase. Nucleic acid amplification based assays involve the use of oligonucleotides or oligomers based on a Inositol hexakisphosphate kinase or RYK kinase nucleic acid sequence to detect transformants containing DNA or RNA encoding Inositol hexakisphosphate kinase or RYK kinase. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of Inositol hexakisphosphate kinase or RYK kinase, using either polyclonal or monoclonal antibodies specific for the protein are known in the art. Examples include enzyme-linked immunosorbent assay (ELISA), radio-immunoassay (RIA), and fluorescence activated cell sorting (FACS). A two-site, monoclonal-based immunoassay utilising monoclonal antibodies reactive to two non-interfering epitopes on Inositol hexakisphosphate kinase or Drl kinase is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labelled hybridisation or PCR probes for detecting sequences related to Inositol hexakisphosphate kinase or RYK kinase polynucleotides include oligo-labelling, nick translation, end-labelling or PCR amplification using a labelled nucleotide.

Alternatively, a Inositol hexakisphosphate kinase or RYK kinase sequence, or any portion thereof may be cloned into a vector for the production of an mRNA probe. Such vectors are known in the art, are commercially available, and may be used to synthesise RNA probes in vitro by addition of an appropriate RNA polymerase such as T7, T3, or SP6 and labelled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding Inositol hexakisphosphate kinase or RYK kinase may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode Inositol hexakisphosphate kinase or RYK kinase may be designed to contain signal sequences, which direct secretion of Inositol hexakisphosphate kinase or RYK kinase through a prokaryotic or eukaryotic cell membrane. Other recombinant constructions may be used to join sequences encoding Inositol hexakisphosphate kinase or RYK kinase to nucleotide sequence encoding a polypeptide domain, which will facilitate purification of soluble proteins. Such purification facilitating domains include, but are not limited to, metal chelating peptides such as histidine-tryptophan modules that allow purification on immobilised metals, protein A domains that allow purification on immobilised immunoglobulin, and the domain utilised in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and Inositol hexakisphosphate kinase or RYK kinase may be used to facilitate purification.

Diagnostics and Therapeutics

The data disclosed in this invention show that the nucleic acids and proteins of the invention and effector molecules thereof are useful in diagnostic and therapeutic applications implicated, for example but not limited to, in metabolic disorders such as obesity as well as related disorders such as metabolic syndrome, eating disorder, cachexia, diabetes mellitus, hypertension, coronary heart disease, hypercholesterolemia, dyslipidemia, osteoarthritis, and gallstones. Hence, diagnostic and therapeutic uses for the Inositol hexakisphosphate kinase or RYK kinase nucleic acids and proteins of the invention are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in diagnostic and therapeutic applications implicated in various applications as described below. For example, but not limited to, cDNAs encoding the Inositol hexakisphosphate kinase or RYK kinase proteins of the invention and particularly their human homologues may be useful in gene therapy, and the Inositol hexakisphosphate kinase or RYK kinase proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids or fragments thereof, may further be useful in diagnostic applications, wherein the presence or amount of the nucleic acids or the proteins are to be assessed. These materials are further useful in the generation of antibodies that bind immunospecifically to the novel substances of the invention for use in therapeutic or diagnostic methods.

For example, in one aspect, antibodies which are specific for Inositol hexakisphosphate kinase or RYK kinase may be used directly as an antagonist, or indirectly as a targeting or delivery mechanism for bringing a pharmaceutical agent to cells or tissue which express Inositol hexakisphosphate kinase or RYK kinase. The antibodies may be generated using methods that are well known in the art. Such antibodies may include, but are not limited to, polyclonal, monoclonal, chimeric, single chain, Fab fragments, and fragments produced by a Fab expression library. Neutralising antibodies (i.e. those which inhibit dimer formation) are especially preferred for therapeutic use.

For the production of antibodies, various hosts including goats, rabbits, rats, mice, humans, and others, may be immunised by injection with Inositol hexakisphosphate kinase or RYK kinase any fragment or oligopeptide thereof which has immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to Inositol hexakisphosphate kinase or RYK kinase have an amino acid sequence consisting of at least five amino acids, and more preferably at least 10 amino acids.

Monoclonal antibodies to Inositol hexakisphosphate kinase or RYK kinase may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce Inositol hexakisphosphate kinase or RYK kinase-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Fragments of antibodies against Inositol hexakisphosphate kinase or RYK kinase, which contain specific binding sites for Inositol hexakisphosphate kinase or RYK kinase, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between Inositol hexakisphosphate kinase or RYK kinase and its specific antibody. A two-site, monoclonal-based immunoassay utilising monoclonal antibodies reactive to two non-interfering Inositol hexakisphosphate kinase or RYK kinase epitopes is preferred, but a competitive binding assay may also be employed (Maddox, supra).

In another embodiment of the invention, the polynucleotides encoding Inositol hexakisphosphate kinase or RYK kinase, or any fragment thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a protein of the invention and modulating its activity, may be generated by a screening and selection procedure involving the use of combinatorial nucleic acid libraries.

In a further aspect, antisense molecules to the polynucleotide encoding Inositol hexakisphosphate kinase or RYK kinase may be used in situations in which it would be desirable to block the transcription of the mRNA. In particular, cells may be transformed with sequences complementary to polynucleotides encoding Inositol hexakisphosphate kinase or RYK kinase. Thus, antisense molecules may be used to modulate Inositol hexakis-phosphate kinase or RYK kinase activity, or to achieve regulation of gene function. Such technology is now well know in the art, and sense or antisense oligomers or larger fragments, can be designed from various locations along the coding or control regions of sequences encoding Inositol hexakisphosphate kinase or RYK kinase. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of a gene encoding Inositol hexakisphosphate kinase or RYK kinase. These techniques are described both in Sambrook et al. (supra) and in Ausubel et al. (supra). Genes encoding Inositol hexakisphosphate kinase or RYK kinase can be turned off by transforming a cell or tissue with expression vectors which express high levels of polynucleotide or fragment thereof which encodes Inositol hexakisphosphate kinase or RYK kinase. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, DNA, RNA, or nucleic acid analogues such as PNA, to the control regions of a gene encoding Inositol hexakisphosphate kinase or RYK kinase, i.e. the promoters, enhancers, and introns. Oligonucleotides derived from the transcription initiation site, e.g. between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding Inositol hexakisphosphate kinase or RYK kinase. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding Inositol hexakisphosphate kinase or RYK kinase. Such DNA sequences may be incorporated into a variety of vectors with suitable RNA polymerase promoters such as T7 or SP6. Alternatively, these cDNA constructs that synthesise antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of Inositol hexakisphosphate kinase or RYK kinase, antibodies to Inositol hexakisphosphate kinase or RYK kinase, mimetics, agonists, antagonists, or inhibitors of Inositol hexakisphosphate kinase or RYK kinase. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g. by means of conventional mixing, dissolving, granulating, dragee-making, levigating, emulsifying, encapsulating, entrapping, or lyophilising processes. After pharmaceutical compositions have been prepared, they can be-placed in an appropriate container and labelled for treatment of an indicated condition. For administration of Inositol hexakisphosphate kinase or RYK kinase, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g. of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example Inositol hexakisphosphate kinase or RYK kinase or fragments thereof, or antibodies against Inositol hexakisphosphate kinase or RYK kinase, which is effective against a specific condition. Therapeutic efficacy and toxicity may be determined by standard pharmaceutical procedures in cell cultures or experimental animals, e.g. ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage being employed, the sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind Inositol hexakisphosphate kinase or RYK kinase may be used for the diagnosis of conditions or diseases characterised by or associated with over- or underexpression of Inositol hexakisphosphate kinase or RYK kinase, or in assays to monitor patients being treated with Inositol hexakisphosphate kinase or RYK kinase, agonists, antagonists or inhibitors. The antibodies useful for diagnostic purposes may be prepared in the same manner as those described above for therapeutics. Diagnostic assays for Inositol hexakisphosphate kinase or RYK kinase include methods, which utilise the antibody and a label to detect Inositol hexakisphosphate kinase or RYK kinase in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring Inositol hexakisphosphate kinase or RYK kinase are known in the art and provide a basis for diagnosing altered or abnormal levels of Inositol hexakisphosphate kinase or RYK kinase expression. Normal or standard values for Inositol hexakisphosphate kinase or RYK kinase expression are established by combining body fluids or cell extracts taken from normal mammalian subjects, preferably human, with antibody to Inositol hexakisphosphate kinase or RYK kinase under conditions suitable for complex formation. The amount of standard complex formation may be quantified by various methods, but preferably by photometric means. Quantities of Inositol hexakisphosphate kinase or RYK kinase expressed in control and disease, samples, e.g. from biopsied tissues are compared with the standard values. Deviation between standard and subject values establishes the parameters for diagnosing disease. Analysis of kinase expression could also be performed by determination of Inositol hexakisphosphate kinase or RYK kinase activity in assay formats well known in the art and described in more detail below.

In another embodiment of the invention, the Inositol hexakisphosphate kinase or RYK kinase polynucleotides may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of Inositol hexakisphosphate kinase or RYK kinase may be correlated with disease. The diagnostic assay may be used to distinguish between absence, presence, and excess expression of Inositol hexakisphosphate kinase or RYK kinase, and to monitor regulation of Inositol hexakisphosphate kinase or RYK kinase levels during therapeutic intervention.

In one aspect, hybridisation with probes which are capable of detecting polynucleotide sequences, including genomic sequences, encoding Inositol hexakisphosphate kinase or RYK kinase or alleles thereof or closely related molecules, may be used to identify nucleic acid sequences which encode Inositol hexakisphosphate kinase or RYK kinase. The specificity of the probe, whether it is made from a highly specific region, e.g. unique nucleotides in the 5′ regulatory region, or a less specific region, e.g. especially in the 3′ coding region, and the stringency of the hybridisation or amplification (maximal, high, intermediate, or low) will determine whether the probe identifies only naturally occurring sequences encoding Inositol hexakisphosphate kinase or RYK kinase, alleles, or related sequences. Probes may also be used for the detection of related sequences, and should preferably contain at least 50% of the nucleotides from any of the Inositol hexakisphosphate kinase or RYK kinase encoding sequences. The hybridisation probes of the subject invention may be DNA or RNA and derived from the nucleotide sequence of a polynucleotide comprising (i) Drosophila CG10082, or IHKPK3, IHKPK1, or IHKPK2, or (ii) Drosophila dnt CG17559, or human RYK kinase, or from a genomic sequence including promoter, enhancer elements, and introns of the naturally occurring Inositol hexakisphosphate kinase or RYK kinase gene. Means for producing specific hybridisation probes for DNAs encoding Inositol hexakisphosphate kinase or RYK kinase include the cloning of nucleic acid sequences encoding Inositol hexakisphosphate kinase or RYK kinase derivatives into vectors for the production of mRNA probes. Such vectors are known in the art, commercially available, and may be used to synthesise RNA probes in vitro by means of the addition of the appropriate RNA polymerases and the appropriate labelled nucleotides. Hybridisation probes may be labelled by a variety of reporter groups; for example, radionuclides such as ³²P or ³⁵S, or enzymatic labels, such as alkaline phosphatase coupled to the probe via avidin/biotin coupling systems, and the like.

Polynucleotide sequences specific for Inositol hexakisphosphate kinase or RYK kinase may be used for the diagnosis of conditions or diseases, which are associated with expression of Inositol hexakisphosphate kinase or RYK kinase. Examples of such conditions or diseases include, but are not limited to, pancreatic diseases and disorders, including diabetes. Polynucleotide sequences specific for Inositol hexakisphosphate kinase or RYK kinase may also be used to monitor the progress of patients receiving treatment for pancreatic diseases and disorders, including diabetes. The polynucleotide sequences encoding Inositol hexakisphosphate kinase or RYK kinase may be used in Southern or Northern analysis, dot blot, or other membrane-based technologies; in PCR technologies; or in dip stick, pin, ELISA or chip assays utilising fluids or tissues from patient biopsies to detect altered Inositol hexakisphosphate kinase or RYK kinase expression. Such qualitative or quantitative methods are well known in the art.

In a particular aspect, the nucleotide sequences encoding Inositol hexakisphosphate kinase or RYK kinase may be useful in assays that detect activation or induction of various metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones. The nucleotide sequences encoding Inositol hexakisphosphate kinase or RYK kinase may be labelled by standard methods, and added to a fluid or tissue sample from a patient under conditions suitable for the formation of hybridisation complexes. After a suitable incubation period, the sample is washed and the signal is quantitated and compared with a standard value. The presence of altered levels of nucleotide sequences encoding Inositol hexakisphosphate kinase or RYK kinase in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of Inositol hexakisphosphate kinase or RYK kinase, a normal or standard profile for expression is established. This may be accomplished by combining body fluids or cell extracts taken from normal subjects, either animal or human, with a sequence, or a fragment thereof, which encodes Inositol hexakisphosphate kinase or RYK kinase, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the metabolic, e.g. pancreatic diseases and disorders.

Additional diagnostic uses for oligonucleotides designed from the sequences encoding Inositol hexakisphosphate kinase or RYK kinase may involve the use of PCR. Such oligomers may be chemically synthesised, generated enzymatically, or produced from a recombinant source. Oligomers will preferably consist of two nucleotide sequences, one with sense orientation (5′.fwdarw.3′) and another with antisense (3′.rarw.5′), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of Inositol hexakisphosphate kinase or RYK kinase include radiolabelling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or colorimetric response gives rapid quantification.

In another embodiment of the invention, the nucleic acid sequences, specific for Inositol hexakisphosphate kinase or RYK kinase, may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding Inositol hexakisphosphate kinase or RYK kinase on a physical chromosomal map and a specific disease, or predisposition to a specific disease, may help to delimit the region of DNA associated with that genetic disease.

The nucleotide sequences of the subject invention may be used to detect differences in gene sequences between normal, carrier, or affected individuals. In situ hybridisation of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localised by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, the proteins, their catalytic or immunogenic fragments or oligopeptides thereof, an in vitro model, a genetically altered cell or animal, can be used for screening libraries of compounds in any of a variety of drug screening techniques. One can identify effectors, e.g. receptors, enzymes, proteins, ligands, or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between the protein and the agent tested, may be measured. Agents could also, either directly or indirectly, influence the activity of the proteins of the invention.

In vivo, the enzymatic kinase activity of the unmodified polypeptides of Inositol hexakisphosphate kinase or RYK kinase, or homologues thereof towards a substrate can be measured. Activation of the kinases may be induced in the natural context by extracellular or intracellular stimuli, such as signaling molecules or environmental influences. One may generate a system containing IHKPK or RYK kinases, or homologues thereof, may it be an organism, a tissue, a culture of cells or cell-free environment, by exogenously applying this stimulus or by mimicking this stimulus by a variety of the techniques, some of them described further below. A system containing activated Inositol hexakisphosphate kinase or RYK kinase, or homologues thereof may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In one embodiment of the invention, one may produce activated Inositol hexakisphosphate kinase or RYK kinase independent of the natural stimuli for the above said purposes by, for example, but not limited to, (i) an agent that mimics the natural stimulus; (ii) an agent, that acts downstream of the natural stimulus, such as activators of the Inositol hexakisphosphate kinase pathway or the RYK kinase, phorbol ester, anisomycin, constitutive active alleles of the Inositol hexakisphosphate kinase or RYK kinase itself as they are described or may be developed; (iii) by introduction of single or multiple amino acid substitutions, deletions or insertions within the sequence of Inositol hexakisphosphate kinase or RYK kinase to yield constitutive active forms; (iv) by the use of isolated fragments of Inositol hexakisphosphate kinase or RYK kinase. In addition, one may generate enzymatically active Inositol hexakisphosphate kinase or RYK kinase in an ectopic system, prokaryotic or eukaryotic, in vivo or in vitro, by co-transfering to this system the activating components.

In addition activity of Inositol hexakisphosphate kinase or RYK kinase against its physiological substrate(s) or derivatives thereof could be measured in cell-based assays. Agents may also interfere with posttranslational modifications of the protein, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from the interacting protein to the protein of the invention, or vice versa, could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interaction are Inositol hexakisphosphate kinase or RYK kinase.

Assays for determining enzymatic activity of the proteins of the invention are well known in the art.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in kinase assays where a kinase substrate such as a protein, a peptide, a lipid, or an organic compound, which may or may not include modifications as further described below, or others are phosphorylated by the proteins or protein fragments of the invention. A therapeutic candidate agent may be identified by its ability to increase or decrease the enzymatic activity of the proteins of the invention. The kinase activity may be detected by change of the chemical, physical or immunological properties of the substrate due to phosphorylation. One example could be the transfer of radioisotopically labelled phosphate groups from an appropriate donor molecule to the kinase substrate catalyzed by the polypeptides of the invention. The phosphorylation of the substrate may be followed by detection of the substrates autoradiography with techniques well known in the art.

In addition the generation of ATP by Inositol hexakisphosphate kinase can be measured by luciferase-dependent bioluminescence generation, an assay principle well known in the art (ATP Determination Kit based on this principle are offered by commercial suppliers).

Yet in another example, the change of mass of the substrate due to its phosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependend selectivity towards the substrate.

Such an analyte could be employed to detect the kinase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the kinase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The kinase substrate may be linked to these or other features with a molecular spacer arm to avoid steric hindrance.

In one example, the kinase substrate may be labelled with a fluorochrome. The binding of the analyte to the labelled substrate in solution may be followed by the technique of fluorescence polarization as it is described in the literature (see, for example, Deshpande, S. et al. (1999) Prog. Biomed. Optics (SPIE) 3603:261; Parker, G. J. et al. (2000) J. Biomol. Screen. 5:77-88; Wu, P. et al. (1997) Anal. Biochem. 249:29-36). In a variation of this example, a fluorescent tracer molecule may compete with the substrate for the analyte to detect kinase activity by a technique which is known to those skilled in the art as indirect fluorescence polarization.

Another technique for drug screening, which may be used, provides for high throughput screening of compounds having suitable binding affinity to the protein of interest as described in published PCT application WO84/03564. In this method, as applied to Inositol hexakisphosphate kinase or RYK kinase large numbers of different small test compounds, e.g. aptamers, peptides, low-moleular weight compounds etc. are synthesised on a solid substrate, such as plastic pins or some other surface. The test compounds are reacted with Inositol hexakisphosphate kinase or RYK kinase, or fragments thereof, and washed. Bound Inositol hexakisphosphate kinase or RYK kinase are then detected by methods well known in the art. Purified Inositol hexakisphosphate kinase or RYK kinase can also be coated directly onto plates for use in the aforementioned drug screening techniques. Alternatively, non-neutralising antibodies can be used to capture the peptide and immobilise it on a solid support.

In another embodiment, one may use competitive drug screening assays in which neutralising antibodies capable of binding Inositol hexakisphosphate kinase or RYK kinase specifically compete with a test compound for binding Inositol hexakisphosphate kinase or RYK kinase. In this manner, the antibodies can be used to detect the presence of any peptide, which shares one or more antigenic determinants with Inositol hexakisphosphate kinase or RYK kinase.

In additional embodiments, the nucleotide sequences which encode Inositol hexakisphosphate kinase or RYK kinase may be used in any molecular biology techniques that have yet to be developed, provided the new techniques rely on properties of nucleotide that are currently known, including, but not limited to, such properties as the triplet genetic code and specific base pair interactions.

The nucleic acids encoding the proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLES), these mice could be used to test whether administration of a candidate modulator alters for example lipid accumulation in the liver, in plasma, or adipose tissues using standard assays well known in the art, such as FPLC, colorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention relates to a kit comprising at least one of
(a) an Inositol hexakisphosphate kinase or RYK kinase nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

The kit may be used for diagnostic or therapeutic purposes or for screening applications as described above. The kit may further containuser instructions.

The Figures show:

FIG. 1 shows the increase of triglyceride content of EP(2)0712 flies caused by homozygous viable integration of the P-vector 5′ into the cDNA of Drosophila gene with GadFly Accession Number CG10082 (column 2; in comparison to controls without integration of this vector, column 1).

FIG. 2 shows the molecular organisation of the mutated inositol hexakisphosphate kinase (Gadfly Accession Number CG10082) gene locus.

FIG. 3 shows the IHKPK sequences

FIG. 3A shows the BLASTP search result for Gadfly Accession Number CG10082 (Query) with the best human homolog matches (Sbject)

FIG. 3B. shows the nucleic acid sequence of human IHKPK3 (SEQ ID NO:1; GenBank Accession Number AF393812)

FIG. 3C. shows the amino acid sequence of human IHKPK3 (SEQ ID NO:2; GenBank Accession Number AAL17053)

FIG. 3D. shows the nucleic acid sequence of human IHKPK1 (SEQ ID NO:3; GenBank Accession Number NM_{—}153273 (formerly GenBank Accession No. BC012944)

FIG. 3E. shows the amino acid sequence of human IHKPK1 (SEQ ID NO:4; GenBank Accession Number NP_{—}695005 (formerly GenBank Accession No. AAH12944)

FIG. 3F. shows the nucleic acid sequence of human IHKPK2 (SEQ ID NO:5; GenBank Accession Number NM_{—}016291)

FIG. 3G. shows the amino acid sequence of human IHKPK2 (SEQ ID NO:6; GenBank Accession Number NP_{—}057375)

FIG. 3H shows a CLUSTAL W (1.82) multiple amino acid sequence alignment of the Drosophila CG10082 (referred to as CG10082_Dm) sequence and the human inositol hexakisphosphate kinase 3 (referred to as IHPK3_Hs), human inositol hexakisphosphate kinase 1 (referred to as IHPK1_Hs), and human inositol hexakisphosphate kinase 2 (referred to as IHPK2_Hs). The alignment was produced using the multisequence alignment program of Clustal W software (Higgins D. G. and Sharp P. M., (1989) CABIOS 5(2): 151-153. Identical amino acid residues are marked with a star.

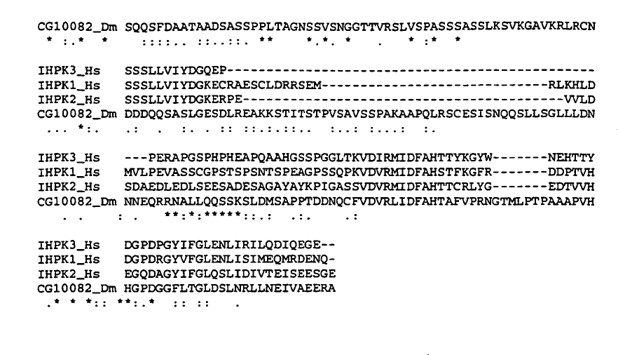

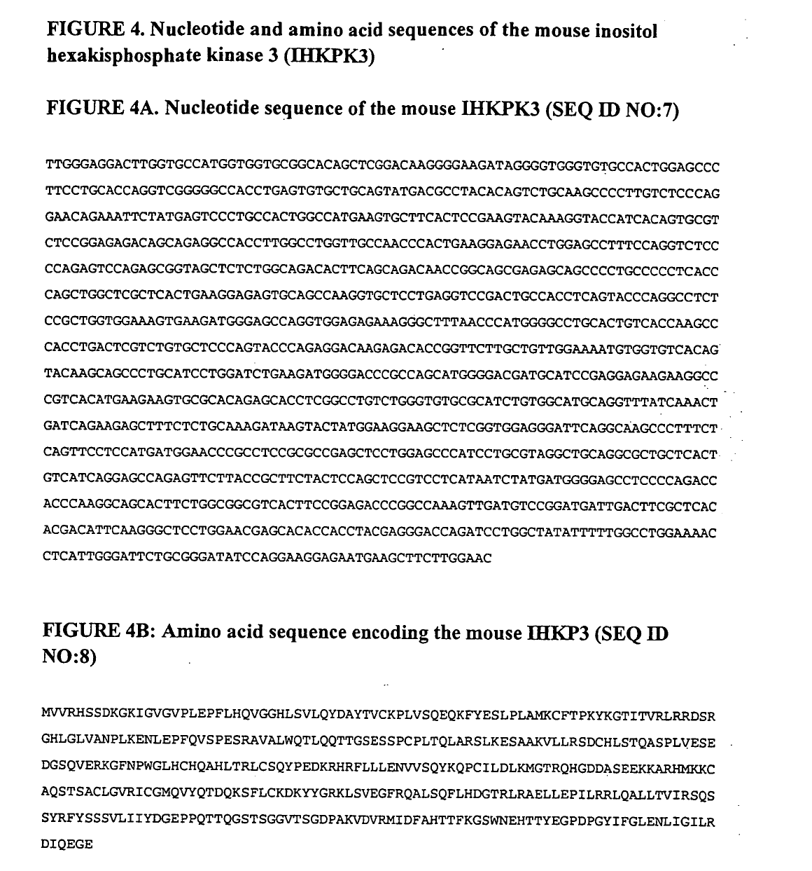

FIG. 4 shows the sequences of the mouse inositol hexakisphosphate kinase 3 (IHKPK3)

FIG. 4A shows the cDNA of the mouse IHKPK3 (SEQ ID NO: 7).

FIG. 4B shows the amino acid sequence (one letter code) encoding the mouse IHKPK3 protein (SEQ ID NO: 8).

FIG. 5-7. Expression of IHKPK in mammalian tissues. The relative RNA-expression is shown on the X-axis. In FIGS. A and B, the tissues tested are given on the Y-axis. “WAT” refers to white adipose tissue, “BAT” refers to brown adipose tissue. In FIG. C, the Y-axis represents the time axis. “d0” refers to day 0 (start of the experiment), “d2”-“d10” refers to day 2-day 10 of adipocyte differentiation).

FIG. 5. Expression of IHKPK3 in mammalian tissues.

FIG. 5A: Real-time PCR analysis of IHKPK3 in wildtype mouse tissues.

FIG. 5B: Real-time PCR mediated analysis of IHKPK3 in different mouse models.

FIG. 5C: Real-time PCR mediated comparison of IHKPK3 expression during the differentiation of 3T3-L1 cells from preadipocytes to mature adipocytes.

FIG. 6. Expression of IHKPK1 in mammalian tissues.

FIG. 6A: Real-time PCR analysis of IHKPK1 in wildtype mouse tissues.

FIG. 6B: Real-time PCR mediated analysis of IHKPK1 in different mouse models.

FIG. 6C: Real-time PCR mediated comparison of IHKPK1 expression during the differentiation of 3T3-L1 cells from preadipocytes to mature adipocytes.

FIG. 7. Expression of IHKPK2 in mammalian tissues.

FIG. 7A: Real-time PCR analysis of IHKPK2 in wildtype mouse tissues.

FIG. 7B: Real-time PCR mediated analysis of IHKPK2 in different mouse models.

FIG. 7C: Real-time PCR mediated comparison of IHKPK2 expression during the differentiation of 3T3-L1 cells from preadipocytes to mature adipocytes.

FIG. 8A shows the increase of triglyceride content of HD-EP(2)21861 flies caused by homozygous viable integration of the P-vector into the first large intron of doughnut on 2 (in comparison to controls without integration of this vector).

FIG. 8B shows the increase of triglyceride and glycogen contents of a drl (Gadfly Accession Number CG17348) mutant.

FIG. 9 shows the molecular organisation of the mutated dnt (Gadfly Accession Number CG17559) gene locus.

FIG. 10A shows the BLASTP search result for CG17559 (Query) with the best human homolog match (Sbject).

FIG. 10B. shows the nucleic acid sequence of RYK kinase (human RYK receptor-like tyrosine kinase precursor, SEQ ID NO:9; GenBank Accession Number NM_{—}002958)

FIG. 10C. shows the amino acid sequence of RYK kinase (human RYK receptor-like tyrosine kinase precursor, SEQ ID NO:10; GenBank Accession Number NP_{—}002949)

FIG. 11. Expression of RYK kinase in mammalian tissues. Real-time PCR mediated comparison of RYK kinase expression during the differentiation of 3T3-L1 cells from preadipocytes to mature adipocytes. The relative RNA-expression is shown on the X-axis, the days of differentiation are shown on the Y-axis (d0=day 0, start of the experiment, until d10=day 10).

The examples illustrate the invention:

### EXAMPLE 1

### Measurement of Energy Storage Metabolites Content

Mutant flies are obtained from a fly mutation stock collection. The flies are grown under standard conditions known to those skilled in the art. In the course of the experiment, additional feedings with bakers yeast (Saccharomyces cerevisiae) are provided. The average increase of energy storage metabolites (triglyceride and glycogen) content of Drosophila containing the EP-vectors in homozygous viable integration was investigated in comparison to control flies (see FIGS. 1 and 8). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. The glycogen content of the flies extract was determined using Roche (Starch UV-method Cat. No. 0207748) assay by measuring changes in the optical density according to the manufacturer's protocol. As a reference protein content of the same extract was measured using BIO-RAD DC Protein Assay according to the manufacturer's protocol. The assay was repeated several times. The average triglyceride level of all flies of the EP collections (referred to as ‘EP-control’) is shown as 100% in FIGS. 1 and 8.

EP(2)0712 homozygous flies show constantly a higher triglyceride content than the controls (50%; column 2 in FIG. 1). Therefore, the loss of gene activity in the locus 57F6 on chromosome 2R where the EP-vector of HD-EP(2)0712 flies is homozygous viable integrated, is responsible for changes in the metabolism of the energy storage triglycerides. Even heterozygous integration of this vector into the cDNA of CG10082 mildly elvevates the triglyceride content.

HD-EP(2)21861 homozygous flies show constantly a higher triglyceride content than the controls (70%; column 2 in FIG. 8A). Therefore, the loss of gene activity in the locus 37D4-37D6 on chromosome 2L where the EP-vector of HD-EP(2)21861 flies is homozygous viable integrated, is responsible for changes in the metabolism of the energy storage triglycerides, therefore representing an model for obese flies.

HD-EP21688 homozygous flies show constantly a higher triglyceride content (than the controls (column 3 in FIG. 8B). HD-EP21688 homozygous flies also show a higher glycogen content than the controls (column 5 in FIG. 8B). The average glycogen level of an internal assay control consisting of two different wildtype strains and an inconspicuous EP-line of the HD stock collection is shown as 100% in the fourth column in FIG. 8B. Standard deviations of the measurements are shown as thin bars. Therefore, the loss of gene activity is responsible for changes in the metabolism of the energy storage metabolites.

### EXAMPLE 2

### Identification of Drosophila Genes and Proteins Associated with Metabolic Control

In FIG. 2, genomic DNA sequence is represented by the assembly as a dotted black line (from position 16568000 to 16581500 on chromosome 2R) including the integration sites of vector for line EP(2)0712 and in FIG. 9, genomic DNA sequence is represented by the assembly as a dotted black line (from position 19160000 to 19195000 on chromosome 2L) including the integration sites of vector for line HD-EP(2)21861. Transcribed DNA sequences (ESTs) and predicted exons are shown as bars in the lower two lines. Predicted exons of the cDNA with GadFly Accession Number CG10082 (FIG. 2) or GadFly Accession Number CG17559 (FIG. 9) are shown as dark grey bars and introns as light grey bars.

Inositol hexakisphosphate kinase encodes for a gene that is predicted by GadFly sequence analysis programs as Accession Number CG10082. dnt encodes for a gene that is predicted by GadFly sequence analysis programs as Accession Number CG17559. Public DNA sequence databases (for example, NCBI GenBank) were screened thereby identifying the corresponding integration sites that are causing an increase of triglyceride content. For example, EP(2)0712 is integrated 5′ in antisense orientation of the cDNA with Accession Number CG10082. In another example, HD-EP(2)21861 is integrated into the first large intron of doughnut on 2 (dnt) in antisense orientation of the cDNA with Accession Number CG17559. Therefore, expression of the cDNA encoding Accession Number CG10082 or CG17559 could be effected by homozygous integration of vectors, leading to increase of the energy storage triglycerides.

### EXAMPLE 3

### Identification of Human Inositol Hexakisphosphate Kinase or RYK Kinase Homologues

Inositol hexakisphosphate kinase or RYK kinase homologous proteins and nucleic acid molecules coding therefore are obtainable from insect or vertebrate species, e.g. mammals or birds. Particularly preferred are nucleic acids and polypeptides encoded thereby of Drosophila CG10082, human inositol hexakisphosphate kinase 3 (GenBank Accession Number AF393812 for the cDNA, AAL17053 for the protein, SEQ ID NO 1 and 2; FIGS. 3B and 3C, respectively), human inositol hexakisphosphate kinase 1 (GenBank Accession Number NM_{—}153273 for the cDNA, NP_{—}695005 for the protein, SEQ ID NO:3 and 4, FIGS. 3D and 3E, respectively), inositol hexakisphosphate kinase 2 (GenBank Accession Number NM_{—}016291 for the cDNA, NP_{—}057375 for the protein, SEQ ID NO:5 and 6, FIGS. 3F and 3G, respectively), or of Drosophila dnt CG17559, human RYK kinase (GenBank Accession Number NM_{—}002958 for the cDNA, NP_{—}002949 for the protein, SEQ ID NO 9 and 10; FIGS. 10B and C, respectively).

As shown in FIG. 3A, the gene product of Drosophila CG10082 is 45% homologous to human IHKPK2 (Accession Number XM_{—}030060.1), which is encoded by sequences located between nucleotide 21459 and 29259 on human chromosome 3 (Accession Number NT_{—}005990), and 51% homologous to human KIAA0263 protein (GenBank Accession Number XP_{—}055065). As shown in FIG. 10A, Drosophila CG17559 gene product is 55% homologous to human tyrosine-protein kinase RYK precursor (GenBank Accession Number NP_{—}002949) (herein referred to as RYK kinase).

### EXAMPLE 4

### Cloning of the Mouse IHKPK3

The Mus musculus IHKPK3 was cloned by polymerase chain reaction on the mouse cDNA clone (EST-clone) image: 336155 (ResGen Invitrogen Corporation) using proofreading DNA-Polymerase (Pfu Turbo, Stratagene) according to standard procedures known to those skilled in the art.

The following primers were used: mIHKPK3.for(attB1-Primer) (SEQ ID NO:11): 5′ GGGG ACA AGT TTG TAC AAA AAA GCA GGC T CTCGAG TT GGG AGG ACT TGG TGC CAT G 3′ and mIHKPK3.rev(attB2-Primer) (SEQ ID NO:12): 5′ GGGG AC CAC TTT GTA CAA GAA AGC TGG GT CTCGAG G TTC CAA GAA GCT TCA TTC TCC T 3′. The primers contain a Gateway extension (in bold, Invitrogen) for further cloning.

### EXAMPLE 5

### Expression of the Polypeptides in Mammalian Tissues

For analyzing the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 per cent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler et al. J Clin Invest July 1993;92(1):272-80, Mizuno et al. Proc Natl Acad Sci U S A Apr. 16, 1996;93(8):3434-8). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

Expression Analysis of the Proteins of the Invention

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH-Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

For the analysis of the expression of the transcripts of the proteins of the invention, taqman analysis was performed using the following primer/probe pairs:

For the amplification of IHKPK3:

Mouse IHKPK 3 forward primer (Seq ID NO: 13) 5′-AGC TTC TCC CTC TTG CCT TCC-3′; Mouse IHKPK 3 reverse primer (Seq ID NO: 14) 5′-GTC CGA GCT GTG CCG C-3′; Mouse IHKPK 3 Taqman probe (Seq ID NO: 15) (5/6-FAM) TGG GAG GAC TTG GTG CCA TGG TG (5/6-TAMRA)

30 For the amplification of IHKPK1:

Mouse IHKPK 1 forward primer (Seq ID NO: 16) 5′-GCA CAC AGC ACA TTC AAG GG-3′; Mouse IHKPK 1 reverse primer (Seq ID NO: 17) 5′-AGC CTC TGT CTG GCC CAT C-3′; Mouse IHKPK 1 Taqman probe (Seq ID NO: 18) (5/6-FAM) TTC CGG GAT GAC CCC ACT GTT CA (5/6-TAMRA)

For the amplification of IHKPK2:

Mouse IHKPK 2 forward primer (Seq ID NO: 19) 5′-GGT GCA GGG CTT CAA GGA G-3′; Mouse IHKPK 2 reverse primer (Seq ID NO: 20) 5′-AGC TCA CGG CGC AGG TAC-3′; Mouse IHKPK 2 Taqman probe (Seq ID NO: 21) (5/6-FAM) CGC TTT TCC AGT TCT TTC ACA ATG GGC (5/6-TAMRA) For the amplification of RYK kinase:

Mouse RYK kinase forward primer (Seq ID NO: 22) 5′-TGG GAG CCT ACG TCT GAC TTC-3′; Mouse RYK kinase reverse primer (Seq ID NO: 23) 5′-CAT CCG TGA CAG ACA GGC AC-3′; Mouse RYK kinase Taqman probe (Seq ID NO: 24) (5/6-FAM) CTC CCA GCT CCG CCA CTC AGA AGA (5/6-TAMRA)

Expression profiling studies confirm the particular relevance of IHKPK3, IHKPK1, IHKPK2, and RYK kinase as regulator of energy metabolism in mammals. The results are shown in FIGS. 5A, 6A, and 7A. IHKPK1 and IHKPK2 proteins show higher expression compared to IHKPK3. IHKPK3 shows its highest expression levels in muscle and heart tissues. In addition, significant expression levels of IHPKK3 were found in metabolic active tissues like white adipocyte tissue (WAT) and brown adipocyte tissue (BAT), (FIG. 5A), confirming a role in the regulation of energy homeostasis and thermogenesis.

Further, we show that IHKPK3, IHKPK1, and IHKPK2 are regulated by fasting and by genetically induced obesity, and that thus the expression of IHKPK3, IHKPK1, and IHKPK2 is under metabolic control. In this invention, we used mouse models of insulin resistance and/or diabetes, such as mice carrying gene knockouts in the leptin pathway (for example, ob (leptin) or db (leptin receptor/ligand) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569). For example, in genetically induced obese (ob/ob) mice, expression of IHKPK3 is strongely induced in BAT and liver. In addition, expression in WAT is severely reduced of wildtype levels in fasted mice. A very strong induction of IHKPK 3 in liver is also noted in fasted mice (FIG. 5B).

The IHKPK proteins were also examined in the in vitro differentiation models for the conversion of pre-adipocytes to adipocytes, as described above. As shown in FIG. 5C, IHKPK3 shows a strong induction of its expression during differentiation, starting on day 8 of differentiation in 3T3-L1 cells (up to 6-fold increase of expression; as shown in FIG. 5C). As shown in FIG. 6C, the expression of IHKPK 1 is strongly downregulated during differentiation, starting on day 4 of differentiation in 3T3-L1 cells. The expression of IHKPK 2 is also significantly downregulated during differentiation, starting on day 8 of differentiation in 3T3-L1 cells (as shown in FIG. 7C).

Taqman analysis revealed that RYK kinase is ubiquitously expressed (data not shown). RYK kinase is expressed in several cell culture model systems of adipocyte differentiation. During the differentiation of 3T3-L1 cells, the level of expression of RYK kinase is decreased during the progression of these preadipocytes to mature adipocytes (FIG. 11).

### EXAMPLE 6

### In Vitro Assays for the Determination of Triglyceride Storage, Synthesis and Transport

Obesity is known to be caused by different reasons such as non-insulin dependent diabetes, increase in triglycerides, increase in carbohydrate bound energy and low energy expenditure. For example, an increase in energy expenditure (and thus, lowering the body weight) would include the elevated utilization of both circulating and intracellular glucose and triglycerides, free or stored as glycogen or lipids as fuel for energy and/or heat production. The cellular level of triglycerides and glycogen is analyzed in cells overexpressing the protein of the invention.

Preparation of Cell Lysates for Analysis of Metabolites

Starting at confluence (d0), cell media was changed every 48 hours. Cells and media were harvested 8 hours prior to media change as follows. Media was collected, and cells were washed twice in PBS prior to lyses in 600 μl HB-buffer (0.5% polyoxyethylene 10 tridecylethane, 1 mM EDTA, 0.01M NaH₂PO₄, pH 7.4). After inactivation at 70° C. for 5 minutes, cell lysates were prepared on Bio 101 systems lysing matrix B (0.1 mm silica beads; Q-Biogene, Carlsbad, USA) by agitation for 2×45 seconds at a speed of 4.5 (Fastprep FP120, Bio 101 Thermosavant, Holbrock, USA). Supernatants of lysed cells were collected after centrifugation at 3000 rpm for 2 minutes, and stored in aliquots for later analysis at −80° C.

Changes in Cellular Triglyceride Levels During Adipogenesis

Cell lysates and media were simultaneously analysed in 96-well plates for total protein and triglyceride content using the Bio-Rad DC Protein assay reagent (Bio-Rad, Munich, Germany) according to the manufacturer's instructions and a modified enzymatic triglyceride kit (GPO-Trinder; Sigma) briefly final volumes of reagents were adjusted to the 96-well format as follows: 10 μl sample was incubated with 200 μl reagent A for 5 minutes at 37° C. After determination of glycerol (initial absorbance at 540 nm), 50 μl reagent B was added followed by another incubation for 5 minutes at 37° C. (final absorbance at 540 nm). Glycerol and triglyceride concentrations were calculated using a glycerol standard set (Sigma) for the standard curve included in each assay.

Changes in Cellular Glycogen Levels During Adipogenesis

Cell lysates and media were simultaneously analysed in triplicates in 96-well plates for total protein and glycogen content using the Bio-Rad DC Protein assay reagent (Bio-Rad, Munich, Germany) according to the manufacturer's instructions and an enzymatic starch kit from Hoffmann-La Roche (Basel, Switzerland). 10-μl samples were incubated with 20-μl amyloglucosidase solution for 15 minutes at 60° C. to digest glycogen to glucose. The glucose is further metabolised with 100 μl distilled water and 100 μl of enzyme cofactor buffer and 12 μl of enzyme buffer (hexokinase and glucose phosphate dehydrogenase). Background glucose levels are determined by subtracting values from a duplicate plate without the amyloglucosidase. Final absorbance is determined at 340 nm. HB-buffer as blank, and a standard curve of glycogen (Hoffmann-La Roche) were included in each assay. Glycogen content in samples were calculated using a standard curve.

Synthesis of Lipids During Adipogenesis

During the terminal stage of adipogenesis (day 12) cells were analysed for their ability to metabolise lipids. A modified protocol to the method of Jensen et al (2000) for lipid synthesis was established. Cells were washed 3 times with PBS prior to serum starvation in Krebs-Ringer-Bicarbonate-Hepes buffer (KRBH; 134 nM NaCl, 3.5 mM KCl, 1.2 mM KH₂ PO₄, 0.5 mM MgSO₄,1.5 mM CaCl₂, 5 mM NaHCO₃, 10 mM Hepes, pH 7.4), supplemented with 0.1% FCS for 2.5 h at 37° C. For insulin-stimulated lipid synthesis, cells were incubated with 1 μM bovine insulin (Sigma; carrier: 0.005N HCl) for 45 min at 37° C. Basal lipid synthesis was determined with carrier only. ¹⁴C(U)-D-glucose (NEN Life Sciences) in a final activity of 1 μCi/Well/ml in the presence of 5 mM glucose was added for 30 min at 37° C. For the calculation of background radioactivity, 25 μM cytochalasin B (Sigma) was used. All assays were performed in duplicate wells. To terminate the reaction, cells were washed 3 times with ice cold PBS, and lysed in 1 ml 0.1 N NaOH. Protein concentration of each well was assessed using the standard Biuret method (Protein assay reagent; Bio-Rad). Total lipids were separated from aqueous phase after overnight extraction in Insta-Fluor scintillation cocktail (Packard Bioscience) followed by scintillation counting.

Transport and Metabolism of Free Fatty Acids During Adipogenesis

During the terminal stage of adipogenesis (d12) cells were analysed for their ability to transport long chain fatty acid across the plasma membrane. A modified protocol to the method of Abumrad et al (1991) (Proc. Natl. Acad. Sci. USA, 1991: 88; 6008-12) for cellular transportation of fatty acid was established. In summary, cells were washed 3 times with PBS prior to serum starvation. This was followed by incubation in KRBH buffer supplemented with 0.1% FCS for 2.5 h at 37° C. Uptake of exogenous free fatty acids was initiated by the addition of isotopic media containing non radioactive oleate and (³H)oleate (NEN Life Sciences) complexed to serum albumin in a final activity of 1 μCi/Well/ml in the presence of 5 mM glucose for 30 min at room temperature (RT). For the calculation of passive diffusion (PD) in the absence of active transport (AT) across the plasma membrane 20 mM of phloretin in glucose free media (Sigma) was added for 30 min at RT. All assays were performed in duplicate wells. To terminate the active transport 20 mM of phloretin in glucose free media was added to the cells. Cells were lysed in 1 ml 0.1N NaOH and the protein concentration of each well were assessed using the standard Biuret method (Protein assay reagent; Bio-Rad). Esterified fatty acids were separated from free fatty acids using overnight extraction in Insta-Fluor scintillation cocktail (Packard Bioscience) followed by scintillation counting.

### EXAMPLE 7

### Glucose Uptake Assay

For the determination of glucose uptake, cells were washed 3 times with PBS prior to serum starvation in KRBH buffer supplemented with 0.1% FCS and 0.5 mM Glucose for 2.5 h at 37° C. For insulin-stimulated glucose uptake, cells were incubated with 1 microM bovine insulin (Sigma; carrier: 0.005N HCl) for 45 min at 37° C. Basal glucose uptake was determined with carrier only. Non-metabolizable 2-deoxy-3H-D-glucose (NEN Life Science, Boston, USA) in a final activity of 0.4 μCi/Well/ml was added for 30 min at 37° C. For the calculation of background radioactivity, 25 μM cytochalasin B (Sigma) was used. All assays were performed in duplicate wells. To terminate the reaction, cells were washed 3 times with ice cold PBS, and lysed in 1 ml 0.1N NaOH. Protein concentration of each well was assessed using the standard Biuret method (Protein assay reagent; Bio-Rad), and scintillation counting of cell lysates in 10 volumes Ultima-gold cocktail (Packard Bioscience, Groningen, Netherlands) was performed.

### EXAMPLE 8

### Generation and Analysis of Transgenic Mice

Generation of the Transgenic Animals

Mouse cDNA was isolated from mouse brown adipose tissue (BAT) using standard protocols as known to those skilled in the art. The cDNA was amplified by RT-PCR and point mutations were introduced into the cDNA.

The resulting mutated cDNA was cloned into a suitable transgenic expression vector. The transgene was microinjected into the male pronucleus of fertilized mouse embryos (preferably strain C57/BL6/CBA F1 (Harlan Winkelmann). Injected embryos were transferred into pseudo-pregnant foster mice. Transgenic founders were detected by PCR analysis. Two independent transgenic mouse lines containing the construct were established and kept on a C57/BL6 background. Briefly, founder animals were backcrossed with C57/BL6 mice to generate F1 mice for analysis. Transgenic mice were continously bred onto the C57/BI6 background. The expression of the proteins of the invention can be analyzed by taqman analysis as described above, and further analysis of the mice can be done as known to those skilled in the art.

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the Inositol hexakisphosphate kinase or RYK kinase gene family or a polypeptide encoded thereby or a fragment or a variant of said nucleic acid molecule or said polypeptide, or an effector of said nucleic acid molecule or said polypeptide, e.g. an antibody, an aptamer or another receptor recognizing a nucleic acid molecule of the Inositol hexakisphosphate kinase or RYK kinase gene family or said polypeptide encoded thereby, preferably together with pharmaceutically acceptable carriers, diluents and/or adjuvants.

2. The composition of claim 1, wherein the nucleic acid molecule is a vertebrate or insect Inositol hexakisphosphate kinase or RYK kinase nucleic acid, particulary encoding human inositol hexakisphosphate kinase (SEQ ID NO:1, SEQ ID NO:3, or SEQ ID NO: 5), human RYK kinase (SEQ ID NO: 9), or a fragment thereof or a variant thereof.

3. The composition of claim 1 or 2, wherein said nucleic acid molecule
(a) hybridizes at 50° C. in a solution containing 1×SSC and 0.1% SDS to a nucleic acid molecule as defined in claim 2 and/or a nucleic acid molecule which is complementary thereto;
(b) it is degenerate with respect to the nucleic acid molecule of (a);
(c) encodes a polypeptide which is at least 85%, preferably at least 90%, more preferably at least 95%, more preferably at least 98% and up to 99.6% identical to human inositol hexakisphosphate kinase 3 (SEQ ID NO:2), human inositol hexakisphosphate kinase 1 (SEQ ID NO:4), human inositol hexakisphosphate kinase 2 (SEQ ID NO:6), or human RYK kinase (SEQ ID NO:10), as defined in claim 2;
(d) differs from the nucleic acid molecule of (a) to (c) by mutation and wherein said mutation causes an alteration, deletion, duplication or premature stop in the encoded polypeptide.

4. The composition of any one of claims 1-3, wherein the nucleic acid molecule is a DNA molecule, particularly a cDNA or a genomic DNA.

5. The composition of any one of claims 1-4, wherein said nucleic acid encodes a polypeptide contributing to regulating the energy homeostasis and/or the metabolism of triglycerides.

6. The composition of any one of claims 1-5, wherein said nucleic acid molecule is a recombinant nucleic acid molecule.

7. The composition of any one of claims 1-6, wherein the nucleic acid molecule is a vector, particularly an expression vector.

8. The composition of any one of claims 1-5, wherein the polypeptide is a recombinant polypeptide.

9. The composition of claim 8, wherein said recombinant polypeptide is a fusion polypeptide.

10. The composition of any one of claims 1-7, wherein said nucleic acid molecule is selected from hybridization probes, primers and anti-sense oligonucleotides.

11. The composition of any one of claims 1-10 which is a diagnostic composition.

12. The composition of any one of claims 1-10 which is a therapeutic composition.

13. The composition of any one of claims 1-12 for the manufacture of an agent for detecting and/or verifying, for the treatment, alleviation and/or prevention of an disorders, including metabolic diseases such as obesity and other body-weight regulation disorders as well as related disorders such as metabolic syndrome, eating disorder, cachexia, diabetes mellitus, hypertension, coronary heart disease, hyper-cholesterolemia, dyslipidemia, osteoarthritis, gallstones and others, in cells, cell masses, organs and/or subjects.

14. Use of a nucleic acid molecule of the Inositol hexakisphosphate 9 kinase or RYK kinase gene family or a polypeptide encoded thereby or a fragment or a variant of said nucleic acid molecule or said polypeptide or an antibody, an aptamer or another receptor recognizing a nucleic acid molecule of the Inositol hexakisphosphate kinase or RYK kinase gene family or a polypeptide encoded thereby for controlling the function of a gene and/or a gene product which is influenced and/or modified by an Inositol hexakisphosphate kinase or RYK kinase homologous polypeptide.

15. Use of a nucleic acid molecule of the Inositol hexakisphosphate kinase or RYK kinase gene family or a polypeptide encoded thereby or a fragment or a variant of said nucleic acid molecule or said polypeptide or an antibody, an aptamer or another receptor recognizing a nucleic acid molecule of the Inositol hexakisphosphate kinase or RYK kinase gene family or a polypeptide encoded thereby for identifying substances capable of interacting with an Inositol hexakisphosphate kinase or RYK kinase homologous polypeptide.

16. A non-human transgenic animal exhibiting a modified expression of an Inositol hexakisphosphate kinase or RYK kinase homologous polypeptide.

17. The animal of claim 1 6, wherein the expression of the Inositol hexakisphosphate kinase or RYK kinase homologous polypeptide is increased and/or reduced.

18. A recombinant host cell exhibiting a modified expression of an Inositol hexakisphosphate kinase or RYK kinase homologous polypeptide.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with an Inositol hexakisphosphate kinase or RYK kinase homologous polypeptide or a fragment thereof under conditions that allow binding of said (poly)peptides;
(b) removing (poly)peptides which do not bind and
(c) identifying (poly)peptides that bind to said Inositol hexakisphosphate kinase or RYK kinase homologous polypeptide.

21. A method of screening for an agent which modulates the interaction of an Inositol hexakisphosphate kinase or RYK kinase homologous polypeptide with a binding target/agent, comprising the steps of
(a) incubating a mixture comprising
(aa) an Inositol hexakisphosphate kinase or RYK kinase homologous polypeptide, or a fragment thereof;
(ab) a binding target/agent of said Inositol hexakisphosphate kinase or RYK kinase homologous polypeptide or fragment thereof; and
(ac) a candidate agent under conditions whereby said Inositol hexakisphosphate kinase or RYK kinase polypeptide or fragment thereof specifically-binds to said binding target/agent at a reference affinity;
(b) detecting the binding affinity of said Inositol hexakisphosphate kinase or RYK kinase polypeptide or fragment thereof to said binding target to determine an (candidate) agent-biased affinity; and
(c) determining a difference between (candidate) agent-biased affinity and the reference affinity.

22. A method of screening for an agent which modulates the activity of an Inositol hexakisphosphate kinase or RYK kinase homologous polypeptide comprising the steps of
(a) incubating a mixture comprising
(aa) an Inositol hexakisphosphate kinase or RYK kinase homologous polypeptide, or a fragment thereof, and
(ab) a candidate agent under conditions whereby said Inositol hexakisphosphate kinase or RYK kinase polypeptide or fragment thereof has a reference activity,
(b) detecting the activity of said Inositol hexakisphosphate kinase or RYK kinase polypeptide or fragment thereof to determine an (candidate) agent-biased activity and
(c) determining a difference between (candidate) agent-biased activity and the reference activity.

23. A method of producing a composition comprising the (poly)peptide identified by the method of claim 20 or the agent identified by the method of claim 21 or 22 with a pharmaceutically acceptable carrier, diluent and/or adjuvant.

24. The method of claim 23 wherein said composition is a pharmaceutical composition for preventing, alleviating or treating of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones and other diseases and disorders.

25. Use of a (poly)peptide as identified by the method of claim 20 or of an agent as identified by the method of claim 21 or 22 for the preparation of a pharmaceutical composition for the treatment, alleviation and/or prevention of of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones and other diseases and disorders.

26. Use of a nucleic acid molecule of the Inositol hexakisphosphate kinase or RYK kinase family or of a fragment thereof for the preparation of a non-human animal which over- or under-expresses the Inositol hexakisphosphate kinase or RYK kinase gene product.

27. Kit comprising at least one of
(a) an Inositol hexakisphosphate kinase or RYK kinase nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

28. Nucleic acid molecule comprising
(a) the nucleotide sequence as shown in SEQ ID NO: 7 and/or a sequence complementary thereto, or
(b) a sequence which is degenerated with respect to the nucleic acid molecule of (a) or
(c) a fragment of the nucleic acid molecule of (a) or (b) having a length of at least 20 nucleotides.

29. A polypeptide or peptide encoded by the nucleic acid molecule of claim 28.
